(19) United States
(12) Patent Application Publication
(10) Pub. No.: US 2020/0155687 A1
(43) Pub. Date: May 21, 2020

(54) METHODS OF TREATMENT USING AN AMPHETAMINE PRODRUG

(71) Applicant: Shire Pharmaceuticals Inc., Lexington, MA (US)

(72) Inventors: Brigitte A. ROBERTSON, Lexington, MA (US); Norman W. BARTON, Phoenix, MD (US)

(73) Assignee: Shire Pharmaceuticals Inc., Lexington, MA (US)

(21) Appl. No.: 16/603,936

(22) PCT Filed: Apr. 10, 2018
(86) PCT No.: PCT/US18/26973

§ 371 Date: Oct. 9, 2019

Related U.S. Application Data
(60) Provisional application No. 62/483,779, filed on Apr. 10, 2017.
(60) Provisional application No. 62/483,790, filed on Apr. 10, 2017.
(60) Provisional application No. 62/513,016, filed on May 31, 2017.
(60) Provisional application No. 62/513,034, filed on May 31, 2017.
(60) Provisional application No. 62/513,131, filed on May 31, 2017.
(60) Provisional application No. 62/513,107, filed on May 31, 2017.

(51) Int. Cl. A61K 47/54; A61K 31/198; A61K 31/137; A61P 25/26

(52) U.S. Cl. CPC A61K 47/542; A61P 25/26; A61K 31/137; A61K 31/198

(57) ABSTRACT

Disclosed are methods of treatment of various conditions and disorders, and symptoms associated therewith, including, for example, fatigue and/or inattention associated with various conditions and disorders, using homoarginine-amphetamine prodrug and/or conjugate, or salts thereof.

### CROSS-REFERENCE TO RELATED APPLICATIONS

This International Patent Application claims priority to U.S. Provisional Applications Nos. 62/483,779 and 62/483,790, filed on Apr. 10, 2017, and 62/513,016, 62/513,034, 62/513,107 and 62/513,131, filed on May 31, 2017, all of which are herein incorporated by reference in their entirety.

### FIELD OF INVENTION

The present invention relates to methods of treatment of various conditions and disorders, and symptoms associated therewith, using an amphetamine prodrug, or a salt thereof.

### BACKGROUND OF THE INVENTION

Fatigue can be described as the lack of energy and motivation (both physical and cognitive). As fatigue can be a symptom of an underlying condition, the treatment may depend upon the condition that is causing the fatigue, regardless of whether it is physical, psychological, or a combination of the two.

Cognitive dysfunction (also known as cognitive impairment) can be described as associated with an impairment in cognition, including memory [visual memory, verbal memory, short- and long-term memory]), attention (sustained, selective and divided), concentration, comprehension, decision making (logic and reasoning), planning, executive functions, information processing (including auditory, visual, simple, complex, and speed/reaction times, and/or learning. Cognitive dysfunction is not caused by any one disease or condition, nor is it limited to a specific age group, and the treatment may depend upon the condition causing the cognitive dysfunction.

Chronic Fatigue Syndrome (CFS), also known as myalgic encephalomyelitis (ME), is characterized by extreme physical or mental fatigue not relieved by rest. Patients with ME/CFS can also suffer from cognitive dysfunction and/or inattention. The terms CFS, ME, and systemic exertion intolerance disease (SEID) describe a medical condition that manifests with a debilitating phenotype of unknown etiology and for which no clear treatment is available. Both diagnosis and treatment of ME/CFS/SEID are symptomatic. The three core symptoms necessary for diagnosis include disabling fatigue that persists for at least 6 months and results in a greatly diminished ability to perform activities that were easily accomplished before the illness, a worsening of symptoms after physical or mental activity that would not have been problematic before the illness (post-exertional malaise), and unrefreshing sleep.

Cognitive dysfunction is a key symptom of ME/CFS and affects the gamut of cognitive abilities (e.g., memory, attention, and information processing). Cognitive dysfunction in ME/CFS has a significant impact on patients and interferes with work and role functioning. Patient-reported cognitive function was selected for assessment because of the prevalence, severity, and impact of cognitive dysfunction reported by patients with ME/CFS. A review of cognitive impairment in ME/CFS (Shanks et al., 2013) notes that research has highlighted the negative impact of ME/CFS on memory (visual memory, verbal memory, short- and long-term memory), concentration, attention, and simple and complex information processing. Problems with cognitive function have been reported by 85% to 95% of patients with ME/CFS (Komaroff and Buchwald, 1991; Grafman, 1993). For the majority of these patients, cognitive problems significantly interfere with daily functioning, particularly work and school functioning (Shanks et al., 2013).

In addition to profound fatigue and cognitive dysfunction, sufferers have inattentiveness, autoimmune manifestations, pain not caused by injury, and other symptoms that are worsened upon exertion. Tender lymph nodes, sore throat, digestive issues, orthostatic intolerance, chills and night sweats, and allergies/sensitivities to food, color, chemicals, or noise can also occur.

No cure or treatment has been approved for ME/CFS, and the FDA recognizes the disease as serious with an unmet medical need for treatment (Guidance for Industry, ME/CFS 2014). According to the Institute of Medicine (TOM) committee report, approximately 836,000 to 2.5 million Americans are affected by ME/CFS. It is also noted in the report that approximately 90% of people with ME/CFS have yet to be diagnosed, so the true prevalence is unknown. Although ME/CFS can affect either gender, women are more frequently diagnosed than men. The average age of onset is 33 years; however, cases have been reported in patients younger than 10 years and older than 70 years (TOM). Approximately 25% of patients with ME/CFS are bed- or house-bound, leading to an annual 17 to 24 billion dollar economic burden from loss of productivity and high medical costs.

Fatigue and cognitive dysfunction are also a characteristic symptoms of fibromyalgia. Fibromyalgia is a medical condition characterized by chronic widespread pain and a heightened pain response to pressure. Other symptoms include fatigue to a degree that normal activities are affected, sleep problems, and troubles with memory. Some people also report restless legs syndrome, bowel or bladder problems, numbness and tingling, and sensitivity to noise, lights or temperature. Fibromyalgia is frequently associated with depression, anxiety, and posttraumatic stress disorder. Other types of chronic pain are also frequently present.

Fatigue and cognitive dysfunction are also a characteristic symptoms of multiple sclerosis (MS). About 80% of patients with MS report suffering from fatigue. Patients with MS also suffer from inattention.

Fatigue, cognitive dysfunction, and inattention are also associated with major depressive disorder (MDD).

Chemotherapy and radiation therapy often result in fatigue in cancer patients. Cancer survivors are also known to suffer from inattention following cancer treatment, including chemotherapy, radiation therapy, and/or surgery. For example, childhood cancers (e.g., acute lymphocytic leukemia (ALL), malignant brain tumors) and the therapies most commonly employed to treat these cancers (surgical intervention, cranial radiation therapy, and central nervous system (CNS) chemotherapy) often result in neurocognitive effects, particularly inattention. Daly et al., J Pediatric Psychology, 2007,32(9):1111-1126. Inattention following cancer therapy, e.g., surgical intervention, cranial radiation therapy, and/or CNS chemotherapy, may not manifest until months or years after completion of the cancer treatment. Small clinical trials have reported on the administration of the stimulant methylphenidate (MPH) to childhood survivors of cancer who received radiation and/or chemotherapy. The reviewed published trials do not disclose why methylphenidate was selected as the stimulant. It may be because amphetamine has been reported to be associated with a greater incidence of weight loss and anorexia compared to MPH and a concern about anorexia in cancer patients. Arnold L E, J Attention Disorders. 2000;3(4):200-211.

Attention deficits have also been reported following chemotherapy for the treatment of adult cancers. For example, Staat et al. report attention deficits in adult women who received chemotherapy for the treatment of breast cancer. Staat et al., Clin J Oncol Nursing, 2005; 9(6):713-721.

A subset of patients with various types of cancers experience a constellation of cognitive dysfunctions prior to receiving any cancer treatment. These cognitive dysfunctions are most commonly reported in the areas of short-term memory, concentration, attention, and planning. Davis et al., BMC Neurology, 2013;13:153. This constellation of problems can also be referred to as “chemobrain” when it first appears during or after chemotherapy.

Krull et al. Pediatr Blood Cancer. 2011 Dec. 15; 57(7): 1191-1196, disclose differences between inattention following treatment for cancer and attention deficit hyperactivity disorder (ADHD). The symptom pattern in ALL survivors was more focused on inattention than in ADHD patients. In a large, randomized, double-blind, crossover trial with methylphenidate in survivors of pediatric ALL, 45% of the sample demonstrated a positive medication response, a substantially lower response rate than observed in ADHD. Krull et al. states that the phenotype of attention problems in ALL survivors appears to differ from that seen in developmental ADHD, and survivors of childhood ALL are likely to require different treatments compared to those commonly employed with ADHD.

An example of a wakefulness disorder is narcolepsy. Narcolepsy is a long-term neurological disorder that involves a decreased ability to regulate sleep-wake cycles. Narcolepsy Fact Sheet, NINDS. NIH Publication No. 03-1637. Retrieved 19 Aug. 2016. Narcolepsy afflicts both children and adults alike. A wakefulness disorder can also be a result of chemotherapy or radiation therapy.

Alcohol addiction, smoking, and Fragile X Syndrome are often associated with hyperactivity and impulsivity.

Other disorders including metabolic disorders, such as obesity, Prader Willi symptoms, and Type 1 and Type 2 diabetes mellitus are also associated with fatigue, cognitive dysfunction, and/or inattention and may also be treated with, or their symptoms treated with, the disclosed amphetamine pro-drugs.

Thus, there exists an unmet urgent therapeutic need for the prevention and treatment of each of the above-mentioned symptoms or indications.

### SUMMARY OF THE INVENTION

Various non-limiting aspects and embodiments of the invention are described below.

The instant invention is based on the surprising discovery that an amphetamine-homoarginine conjugate, a prodrug of amphetamine, is effective in treating or preventing various conditions and disorders or symptoms of the conditions and disorders, such as chronic fatigue syndrome (CFS), also known as myalgic encephalomyelitis (ME); fatigue, cognitive dysfunction, and/or inattention in a patient suffering from or diagnosed with ME/CFS; fatigue, cognitive dysfunction, and/or inattention in a patient suffering from or diagnosed with fibromyalgia; fatigue, cognitive dysfunction, and/or inattention in a patient suffering from or diagnosed with multiple sclerosis (MS); fatigue, cognitive dysfunction, and/or inattention in a patient suffering from or diagnosed with major depressive disorder (MDD); fatigue, cognitive dysfunction, and/or inattention in a patient suffering from or diagnosed with traumatic brain injury; fatigue, cognitive dysfunction, and/or inattention in a menopausal patient; fatigue, cognitive dysfunction, and/or inattention in a patient suffering from or diagnosed with the negative symptoms of schizophrenia; post-cancer therapy fatigue, cognitive dysfunction, and/or inattention; fatigue in cancer patients caused by chemotherapy, radiation therapy, surgery, or a combination thereof, cognitive dysfunction in cancer patients caused by chemotherapy, radiation therapy, surgery, or a combination thereof; inattention in cancer patients caused by chemotherapy, radiation therapy, surgery, or a combination thereof, wakefulness disorder such as narcolepsy; wakefulness disorder as a result of chemotherapy, radiation therapy, surgery, or a combination thereof; hyperactivity and/or impulsivity associated with alcohol addiction, smoking, and symptoms of the Fragile X Syndrome; and fatigue, cognitive dysfunction, and/or inattention in a patient suffering from or diagnosed with a metabolic disorder, e.g., obesity, appetite related symptoms of the Prader Willi Syndrome, and Type 1 and Type 2 diabetes mellitus.

In one aspect, the present invention provides a method of treating or preventing fatigue which comprises administering to a patient in need thereof a therapeutically effective amount of an amphetamine-homoarginine conjugate or a salt thereof for the treatment of fatigue. These amounts may be the same or different for different indications, combinations of indications, or patient populations. The salt may be any pharmaceutically acceptable salt including, for example, a mesylate, a hydrochloride, sulfate, oxalate, triflate, citrate, malate, tartrate, phosphate, nitrate, benzoate, or a mixture thereof. In one embodiment, the amphetamine-homoarginine conjugate is 1-homoarginine-d-amphetamine dihydrochloride.

In one embodiment, the fatigue is in patients suffering from or diagnosed with ME/CFS. In another embodiment, the fatigue is in patients suffering from or diagnosed with fibromyalgia. In another embodiment, the fatigue is in patients suffering from or diagnosed with MS. In another embodiment, the fatigue is in patients suffering from or diagnosed with MDD. In another embodiment, the fatigue is in patients suffering from or diagnosed with menopause. In another embodiment, the fatigue is in patients suffering from or diagnosed with traumatic brain injury. In another embodiment, the fatigue is in patients suffering from or diagnosed with negative symptoms of schizophrenia. In another embodiment, the fatigue is in patients suffering from or diagnosed with narcolepsy. In another embodiment, the fatigue is in patients suffering from or diagnosed with cancer. In one embodiment, the fatigue is in cancer patients caused by chemotherapy, radiation therapy, surgery, or a combination thereof.

The present invention provides a method of treating or preventing cognitive dysfunction in a patient which comprises administering a therapeutically effective amount of an amphetamine-homoarginine conjugate or a salt thereof to the patient for the treatment of cognitive dysfunction. These amounts may be the same or different for different indications, combinations of indications, or patient populations. The salt may be any pharmaceutically acceptable salt including, for example, a mesylate, a hydrochloride, sulfate, oxalate, triflate, citrate, malate, tartrate, phosphate, nitrate, benzoate, or a mixture thereof. In one embodiment, the amphetamine-homoarginine conjugate is 1-homoarginine-d-amphetamine dihydrochloride.

In another embodiment, the cognitive dysfunction is in patients suffering from or diagnosed with ME/CFS. In another embodiment, the cognitive dysfunction is in patients suffering from or diagnosed with fibromyalgia. In another embodiment, the cognitive dysfunction is in patients suffering from or diagnosed with MS. In another embodiment, the cognitive dysfunction is in patients suffering from or diagnosed with MDD. In another embodiment, the cognitive dysfunction is in patients suffering from or diagnosed with menopause. In another embodiment, the cognitive dysfunction is in patients suffering from or diagnosed with traumatic brain injury. In another embodiment, the cognitive dysfunction is in patients suffering from or diagnosed with negative symptoms of schizophrenia. In another embodiment, the cognitive dysfunction is in patients suffering from or diagnosed with narcolepsy. In another embodiment, the cognitive dysfunction is in patients suffering from or diagnosed with cancer. In one embodiment, the cognitive dysfunction is in cancer patients caused by chemotherapy, radiation therapy, surgery, or a combination thereof.

In one aspect, the present invention provides a method of treating or preventing inattention which comprises administering to a patient in need thereof a therapeutically effective amount of an amphetamine-homoarginine conjugate or a salt thereof for the treatment of inattention. These amounts may be the same or different for different indications, combinations of indications, or patient populations. The salt may be any pharmaceutically acceptable salt including, for example, a mesylate, a hydrochloride, sulfate, oxalate, triflate, citrate, malate, tartrate, phosphate, nitrate, benzoate, or a mixture thereof. In one embodiment, the amphetamine-homoarginine conjugate is 1-homoarginine-d-amphetamine dihydrochloride.

In another embodiment, the inattention is in patients suffering from or diagnosed with ME/CFS. In another embodiment, the inattention is in patients suffering from or diagnosed with fibromyalgia. In another embodiment, the inattention is in patients suffering from or diagnosed with MS. In another embodiment, the inattention is in patients suffering from or diagnosed with MDD. In another embodiment, the inattention is in patients suffering from or diagnosed with menopause. In another embodiment, the inattention is in patients suffering from or diagnosed with traumatic brain injury. In another embodiment, the inattention is in patients suffering from or diagnosed with negative symptoms of schizophrenia. In another embodiment, the inattention is in patients suffering from or diagnosed with narcolepsy. In another embodiment, the inattention is in patients suffering from or diagnosed with cancer. In one embodiment, the inattention is in cancer patients caused by chemotherapy, radiation therapy, surgery, or a combination thereof.

In one aspect, the prevent invention provides a method for the treatment of symptoms related to fatigue and cognitive impairment in patients with chronic fatigue syndrome, also known as myalgic encephalomyelitis (ME/CSF), which comprises administering to a patient in need thereof a therapeutically effective amount of an amphetamine-homoarginine conjugate or a salt thereof. In certain embodiments, the present invention provides a method for the treatment of at least one symptom related to fatigue and cognitive dysfunction in chronic fatigue syndrome, also known as myalgic encephalomyelitis (ME/CSF), which comprises administering to a patient in need thereof a therapeutically effective amount of an amphetamine-homoarginine conjugate or a salt thereof for the treatment of at least one symptom. In certain embodiments, the prevent invention provides a method for the treatment of at least one symptom related to fatigue and cognitive dysfunction in chronic fatigue syndrome, also known as myalgic encephalomyelitis (ME/CSF), which comprises administering to an adult patient in need thereof a therapeutically effective amount of an amphetamine-homoarginine conjugate or a salt thereof for the treatment of at least one symptom.

In one aspect, the prevent invention provides a method to improve symptoms of fatigue (physical symptoms and cognitive symptoms) in patients with myalgic encephalomyelitis/chronic fatigue syndrome (ME/CFS), which comprises administering to a patient in need thereof a therapeutically effective amount of an amphetamine-homoarginine conjugate or a salt thereof. In certain embodiments, the prevent invention provides a method to improve symptoms of fatigue (physical symptoms and cognitive symptoms) in adult patients with myalgic encephalomyelitis/chronic fatigue syndrome (ME/CFS), which comprises administering to a patient in need thereof a therapeutically effective amount of an amphetamine-homoarginine conjugate or a salt thereof. In certain embodiments, the prevent invention provides at a method to improve at least one symptom of fatigue (physical symptoms and/or cognitive symptoms) associated with chronic fatigue syndrome (CFS), also known as myalgic encephalomyelitis (ME), which comprises administering to a patient in need thereof a therapeutically effective amount of an amphetamine-homoarginine conjugate or a salt thereof for the improvement of at least one symptom. In certain embodiments, the prevent invention provides at a method to improve at least one symptom of fatigue (physical symptoms and/or cognitive symptoms) associated with chronic fatigue syndrome (CFS), also known as myalgic encephalomyelitis (ME), which comprises administering to an adult patient in need thereof a therapeutically effective amount of an amphetamine-homoarginine conjugate or a salt thereof for the improvement of at least one symptom.

In another aspect, the present invention provides a method of treating or preventing symptoms associated with chronic fatigue syndrome (CFS), also known as myalgic encephalomyelitis (ME), which comprises administering to a patient in need thereof a therapeutically effective amount of an amphetamine-homoarginine conjugate or a salt thereof. In one embodiment, the present invention provides a method of treating or preventing fatigue, cognitive dysfunction, and/or inattention in a patient with ME/CFS which comprises administering to a patient in need thereof a therapeutically effective amount for the appropriate indication or indications of an amphetamine-homoarginine conjugate or a salt thereof. In one embodiment, the present invention provides a method of treating or preventing fatigue in a patient with ME/CFS which comprises administering to a patient in need thereof a therapeutically effective amount for the treatment of fatigue of an amphetamine-homoarginine conjugate or a salt thereof. In one embodiment, the present invention provides a method of treating or preventing inattention in a patient with ME/CFS which comprises administering to a patient in need thereof a therapeutically effective amount for the treatment of inattention of an amphetamine-homoarginine conjugate or a salt thereof. In one embodiment, the present invention provides a method of treating or preventing cognitive dysfunction in a patient with ME/CFS which comprises administering to a patient in need thereof a therapeutically effective amount for the treatment of cognitive dysfunction of an amphetamine-homoarginine conjugate or a salt thereof. These amounts may be the same or different for different indications, combinations of indications, or patient populations. The salt may be any pharmaceutically acceptable salt including, for example, a mesylate, a hydrochloride, sulfate, oxalate, triflate, citrate, malate, tartrate, phosphate, nitrate, benzoate, or a mixture thereof. In one embodiment, the amphetamine-homoarginine conjugate is 1-homoarginine-d-amphetamine dihydrochloride.

In another aspect, the present invention provides a method of treating or preventing fatigue, cognitive dysfunction and/or inattention in a patient suffering from or diagnosed with fibromyalgia, which comprises administering to a patient in need thereof a therapeutically effective amount for the appropriate indication or indications of an amphetamine-homoarginine conjugate or a salt thereof. In one embodiment, the present invention provides a method of treating or preventing fatigue associated with fibromyalgia which comprises administering to a patient in need thereof a therapeutically effective amount for the treatment of fatigue of an amphetamine-homoarginine conjugate or a salt thereof. In another embodiment, the present invention provides a method of treating or preventing inattention associated with fibromyalgia which comprises administering to a patient in need thereof a therapeutically effective amount for the treatment of inattention of an amphetamine-homoarginine conjugate or a salt thereof. In another embodiment, the present invention provides a method of treating or preventing cognitive dysfunction associated with fibromyalgia which comprises administering to a patient in need thereof a therapeutically effective amount for the treatment of cognitive dysfunction of an amphetamine-homoarginine conjugate or a salt thereof. These amounts may be the same or different for different indications, combinations of indications, or patient populations. The salt may be any pharmaceutically acceptable salt including, for example, a mesylate, a hydrochloride, sulfate, oxalate, triflate, citrate, malate, tartrate, phosphate, nitrate, benzoate, or a mixture thereof. In one embodiment, the amphetamine-homoarginine conjugate is 1-homoarginine-d-amphetamine dihydrochloride.

In another aspect, the present invention provides a method of treating or preventing fatigue, cognitive dysfunction and/or inattention in a patient suffering from or diagnosed with multiple sclerosis (MS), which comprises administering to a patient in need thereof a therapeutically effective amount for the appropriate indication or indications of an amphetamine-homoarginine conjugate or a salt thereof. In one embodiment, the present invention provides a method of treating or preventing fatigue in a patient suffering from or diagnosed with MS, which comprises administering to a patient in need thereof a therapeutically effective amount for the treatment of fatigue of an amphetamine-homoarginine conjugate or a salt thereof. In another embodiment, the present invention provides a method of treating or preventing cognitive dysfunction in a patient suffering from or diagnosed with MS which comprises administering to a patient in need thereof a therapeutically effective amount for the treatment of cognitive dysfunction of an amphetamine-homoarginine conjugate or a salt thereof. In another embodiment, the present invention provides a method of treating or preventing inattention in a patient suffering from or diagnosed with MS which comprises administering to a patient in need thereof a therapeutically effective amount for the treatment of inattention of an amphetamine-homoarginine conjugate or a salt thereof. These amounts may be the same or different for different indications, combinations of indications, or patient populations. The salt may be any pharmaceutically acceptable salt including, for example, a mesylate, a hydrochloride, sulfate, oxalate, triflate, citrate, malate, tartrate, phosphate, nitrate, benzoate, or a mixture thereof. In one embodiment, the amphetamine-homoarginine conjugate is 1-homoarginine-d-amphetamine dihydrochloride.

In another aspect, the present invention provides a method of treating or preventing fatigue, cognitive dysfunction and/or inattention in a patient suffering from or diagnosed with major depressive disorder (MDD), which comprises administering to a patient in need thereof a therapeutically effective amount for the appropriate indication or indications of an amphetamine-homoarginine conjugate or a salt thereof. In one embodiment, the present invention provides a method of treating or preventing fatigue in a patient suffering from or diagnosed with MDD, which comprises administering to a patient in need thereof a therapeutically effective amount for the treatment of fatigue of an amphetamine-homoarginine conjugate or a salt thereof. In another embodiment, the present invention provides a method of treating or preventing cognitive dysfunction in a patient suffering from or diagnosed with MDD, which comprises administering to a patient in need thereof a therapeutically effective amount for the treatment of cognitive dysfunction of an amphetamine-homoarginine conjugate or a salt thereof. In another embodiment, the present invention provides a method of treating or preventing inattention in a patient suffering from or diagnosed with MDD, which comprises administering to a patient in need thereof a therapeutically effective amount for the treatment of inattention of an amphetamine-homoarginine conjugate or a salt thereof. These amounts may be the same or different for different indications, combinations of indications, or patient populations. In an embodiment of the invention, the patient is elderly. The salt may be any pharmaceutically acceptable salt including, for example, a mesylate, a hydrochloride, sulfate, oxalate, triflate, citrate, malate, tartrate, phosphate, nitrate, benzoate, or a mixture thereof. In one embodiment, the amphetamine-homoarginine conjugate is 1-homoarginine-d-amphetamine dihydrochloride.

In one aspect, the present invention provides a method of treating or preventing post-cancer therapy fatigue which comprises administering to a patient in need thereof a therapeutically effective amount for the treatment of fatigue of an amphetamine-homoarginine conjugate or a salt thereof. The amount may be the same or different for different indications, combinations of indications, or patient populations. In various embodiments of the invention, the cancer therapy is chemotherapy, radiation therapy, surgery, or a combination thereof. In one embodiment, the cancer therapy is a combination of at least two of the following: chemotherapy, radiation therapy, and surgery. In various embodiments, the amphetamine-homoarginine conjugate or a salt thereof is initially administered prior to the cancer therapy, after cancer therapy and before the patient is diagnosed with post-cancer therapy fatigue, or after cancer therapy and after the patient is diagnosed with post-cancer therapy fatigue. The salt may be any pharmaceutically acceptable salt including, for example, a mesylate, a hydrochloride, sulfate, oxalate, triflate, citrate, malate, tartrate, phosphate, nitrate, benzoate, or a mixture thereof. In one embodiment, the amphetamine-homoarginine conjugate is 1-homoarginine-d-amphetamine dihydrochloride.

In one aspect, the present invention provides a method of treating or preventing post-cancer therapy cognitive dysfunction which comprises administering to a patient in need thereof a therapeutically effective amount for the treatment of cognitive dysfunction of an amphetamine-homoarginine conjugate or a salt thereof. The amount may be the same or different for different indications, combinations of indications, or patient populations. In various embodiments of the invention, the cancer therapy is chemotherapy, radiation therapy, surgery, or a combination thereof. In one embodiment, the cancer therapy is a combination of at least two of the following: chemotherapy, radiation therapy, and surgery. In various embodiments, the amphetamine-homoarginine conjugate or a salt thereof is initially administered prior to the cancer therapy, after cancer therapy and before the patient is diagnosed with post-cancer therapy cognitive dysfunction, or after cancer therapy and after the patient is diagnosed with post-cancer therapy cognitive dysfunction. The salt may be any pharmaceutically acceptable salt including, for example, a mesylate, a hydrochloride, sulfate, oxalate, triflate, citrate, malate, tartrate, phosphate, nitrate, benzoate, or a mixture thereof. In one embodiment, the amphetamine-homoarginine conjugate is 1-homoarginine-d-amphetamine dihydrochloride.

In another aspect, the present invention provides a method of treating or preventing post-cancer therapy inattention which comprises administering to a patient in need thereof a therapeutically effective amount for the treatment of inattention of an amphetamine-homoarginine conjugate or a salt thereof. The amount may be the same or different for different indications, combinations of indications, or patient populations. In various embodiments of the invention, the cancer therapy is chemotherapy, radiation therapy, surgery, or a combination thereof. In one embodiment, the cancer therapy is a combination of at least two of the following: chemotherapy, radiation therapy, and surgery. In various embodiments, the amphetamine-homoarginine conjugate or a salt thereof is initially administered prior to the cancer therapy, after cancer therapy and before the patient is diagnosed with post-cancer therapy inattention, or after cancer therapy and after the patient is diagnosed with post-cancer therapy inattention. The salt may be any pharmaceutically acceptable salt including, for example, a mesylate, a hydrochloride, sulfate, oxalate, triflate, citrate, malate, tartrate, phosphate, nitrate, benzoate, or a mixture thereof. In one embodiment, the amphetamine-homoarginine conjugate is 1-homoarginine-d-amphetamine dihydrochloride.

In one aspect, the present invention provides a method of treating or preventing a wakefulness disorder which comprises administering to a patient in need thereof a therapeutically effective amount for the treatment of a wakefulness disorder of an amphetamine-homoarginine conjugate or a salt thereof. The amount may be the same or different for different indications, combinations of indications, or patient populations. In one embodiment of the invention, the wakefulness disorder is narcolepsy. In another embodiment, wakefulness disorder is as a result of chemotherapy, surgery, and/or radiation therapy. The salt may be any pharmaceutically acceptable salt including, for example, a mesylate, a hydrochloride, sulfate, oxalate, triflate, citrate, malate, tartrate, phosphate, nitrate, benzoate, or a mixture thereof. In one embodiment, the amphetamine-homoarginine conjugate is 1-homoarginine-d-amphetamine dihydrochloride.

In one aspect, the present invention provides a method of treating or preventing hyperactivity and/or impulsivity which comprises administering to a patient in need thereof a therapeutically effective amount for the appropriate indication or indications of an amphetamine-homoarginine conjugate or a salt thereof. The amount may be the same or different for different indications, combinations of indications, or patient populations. In various embodiments of the invention, the hyperactivity and/or impulsivity may be associated with one or more of alcohol addiction, smoking, and/or symptoms of the Fragile X syndrome. The salt may be any pharmaceutically acceptable salt including, for example, a mesylate, a hydrochloride, sulfate, oxalate, triflate, citrate, malate, tartrate, phosphate, nitrate, benzoate, or a mixture thereof. In one embodiment, the amphetamine-homoarginine conjugate is 1-homoarginine-d-amphetamine dihydrochloride.

In one aspect, the present invention provides a method of treating or preventing fatigue, inattention, and/or cognitive dysfunction associated with metabolic disorders which comprises administering to a patient in need thereof a therapeutically effective amount for the appropriate indication or indications of an amphetamine-homoarginine conjugate or a salt thereof. The amount may be the same or different for different indications, combinations of indications, or patient populations. In various embodiments of the invention, the metabolic disorders may be one or more of obesity, appetite related symptoms of the Prader Willi Syndrome, and Type 1 and Type 2 diabetes mellitus. The salt may be any pharmaceutically acceptable salt including, for example, a mesylate, a hydrochloride, sulfate, oxalate, triflate, citrate, malate, tartrate, phosphate, nitrate, benzoate, or a mixture thereof. In one embodiment, the amphetamine-homoarginine conjugate is 1-homoarginine-d-amphetamine dihydrochloride.

In one embodiment of the present invention, the amphetamine-homoarginine conjugate or a salt of the amphetamine-homoarginine conjugate is initially administered prior to the chemotherapy or radiation therapy.

In an alternative embodiment, the amphetamine-homoarginine conjugate or a salt of the amphetamine-homoarginine conjugate is initially administered after chemotherapy, radiation therapy, or surgery and before the patient is diagnosed with fatigue caused by chemotherapy, radiation therapy, or surgery.

In an alternative embodiment, the amphetamine-homoarginine conjugate or a salt of the amphetamine-homoarginine conjugate is initially administered after chemotherapy, radiation therapy, or surgery and before the patient is diagnosed with a sleep and/or wakefulness disorder caused by chemotherapy, radiation therapy, or surgery.

In a further embodiment, the amphetamine-homoarginine conjugate or a salt of the amphetamine-homoarginine conjugate is initially administered after chemotherapy, radiation therapy, or surgery and after the patient is diagnosed with fatigue caused by chemotherapy, radiation therapy, or surgery.

In an alternative embodiment, the amphetamine-homoarginine conjugate or a salt of the amphetamine-homoarginine conjugate is initially administered after chemotherapy, radiation therapy, or surgery and after the patient is diagnosed with a wakefulness disorder caused by chemotherapy, radiation therapy, or surgery.

In an alternative embodiment, the amphetamine-homoarginine conjugate or a salt of the amphetamine-homoarginine conjugate is initially administered after cancer therapy and before the patient is diagnosed with post-cancer therapy inattention.

In a further embodiment, the amphetamine-homoarginine conjugate or a salt of the amphetamine-homoarginine conjugate is initially administered after cancer therapy and after the patient is diagnosed with post-cancer therapy inattention.

In one aspect, the present invention provides a method of treating a cognitive dysfunction in a patient suffering from or diagnosed with a cancer comprising administering a therapeutically effective amount for cognitive dysfunction of an amphetamine-homoarginine conjugate or a salt of the amphetamine-homoarginine conjugate to a patient in need thereof, wherein the cognitive dysfunction is selected from the group consisting of: a short-term memory problem, an attention problem, a concentration problem, a planning problem, and a combination thereof; and

wherein the cognitive dysfunction appears before the patient receives chemotherapy.

In one aspect, the present invention provides a method of treating a cognitive dysfunction in a patient suffering from or diagnosed with a cancer comprising administering a therapeutically effective amount for cognitive dysfunction of an amphetamine-homoarginine conjugate or a salt of the amphetamine-homoarginine conjugate to a patient in need thereof,

wherein the cognitive dysfunction is selected from the group consisting of: a short-term memory problem, an attention problem, a concentration problem, a planning problem, and a combination thereof; and
wherein the cognitive dysfunction arises during chemotherapy or after the patient receives chemotherapy.

In one aspect, the present invention further provides a method of treating a cognitive dysfunction in a patient suffering from or diagnosed with a cancer comprising administering a therapeutically effective amount for cognitive dysfunction of an amphetamine-homoarginine conjugate or a salt of the amphetamine-homoarginine conjugate to a patient in need thereof, wherein the cognitive dysfunction is selected from the group consisting of: a short-term memory problem, an attention problem, a concentration problem, a planning problem, and a combination thereof wherein the cognitive dysfunction arises during chemotherapy or after the patient receives chemotherapy.

In one aspect, the present invention further provides a method of treating fatigue, cognitive dysfunction and/or inattention in a patient suffering from or diagnosed with traumatic brain injury which comprises administering a therapeutically effective amount for the appropriate indication or indications of an amphetamine-homoarginine conjugate or a salt of the amphetamine-homoarginine conjugate to a patient in need thereof.

In one aspect, the present invention provides a method of treating fatigue, cognitive dysfunction and/or inattention in an elderly patient suffering from or diagnosed with major depressive disorder, which comprises administering a therapeutically effective amount for the appropriate indication or indications of an amphetamine-homoarginine conjugate or a salt of the amphetamine-homoarginine conjugate to a patient in need thereof.

In one aspect, the present invention encompasses a method of treating fatigue, cognitive dysfunction and/or inattention in a menopausal patient, which comprises administering a therapeutically effective amount for the appropriate indication or indications of an amphetamine-homoarginine conjugate or a salt of the amphetamine-homoarginine conjugate to a patient in need thereof.

In one aspect according to the present invention, a method is provided for treating fatigue, cognitive dysfunction and/or inattention in a patient suffering from or diagnosed with the negative symptoms of schizophrenia, which comprises administering a therapeutically effective amount for the appropriate indication or indications of an amphetamine-homoarginine conjugate or a salt of the amphetamine-homoarginine conjugate to a patient in need thereof.

In one aspect, the present invention provides a method of treating or preventing any of the diseases, disorders, and/or symptoms disclosed herein which comprises administering to a patient in need thereof a therapeutically effective amount for the appropriate indication or indications of an amphetamine-homoarginine conjugate or a salt thereof. The salt may be any pharmaceutically acceptable salt including, for example, a mesylate, a hydrochloride, sulfate, oxalate, triflate, citrate, malate, tartrate, phosphate, nitrate, benzoate, or a mixture thereof.

In one aspect, the amphetamine-homoarginine conjugate is 1-homoarginine-d-amphetamine dihydrochloride, which is represented by the following Formula I:

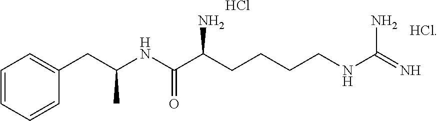

In one aspect, the dose of the conjugate or the salt of the conjugate may be, for example, about 1 mg to about 500 mg; about 5 mg to about 250 mg; about 5 mg to about 150 mg; or about 10 mg to about 100 mg.

These and other aspects of the present invention will become apparent to those skilled in the art after a reading of the following detailed description of the invention, including the appended claims.

### DETAILED DESCRIPTION

Detailed embodiments of the present invention are disclosed herein; however, it is to be understood that the disclosed embodiments are merely illustrative of the invention that may be embodied in various forms. In addition, each of the examples given in connection with the various embodiments of the invention is intended to be illustrative, and not restrictive. Therefore, specific structural and functional details disclosed herein are not to be interpreted as limiting, but merely as a representative basis for teaching one skilled in the art to variously employ the present invention.

Unless defined otherwise, all technical and scientific terms used herein have the same meaning as commonly understood by one of ordinary skill in the art to which this invention belongs.

As used in this specification and the appended claims, the singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure.

The terms “treat” or “treatment” of a state, disorder or condition include: (1) inhibiting the state, disorder or condition, i.e., arresting, reducing or delaying the development of the disease or a relapse thereof or at least one clinical or sub-clinical symptom thereof or (2) relieving the disease, i.e., causing regression of the state, disorder or condition or at least one of its clinical or sub-clinical symptoms. The benefit to a subject to be treated is either statistically significant or at least perceptible to the patient or to the physician.

The terms “prevent” or “preventing” a state, disorder or condition include: preventing, delaying, or reducing the incidence and/or likelihood of the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition.

The terms “improve” or “improvement” as used herein, for example, refers to the improvement of a symptom associated with a disease, disorder, or condition, and can refer to an improvement in at least one parameter measuring or quantitating the symptom. Accordingly, the term encompasses an improvement of about 10%, 20%, 30%, 40%, 50%, 60%, 70%, 80%, 90% or 100% in such parameter.

A “subject” or “patient” or “individual” or “animal”, as used herein, refers to humans, veterinary animals (e.g., cats, dogs, cows, horses, sheep, pigs, etc.) and experimental animal models of diseases (e.g., mice, rats). In an aspect of the invention, a subject according to the present invention is an adult patient. “Adult” as used herein refers to a patient age 18 years or greater. In a further aspect of the invention, a patient is a pediatric patient. In certain embodiments, a pediatric subject can be a human subject from ages of about 6 to about 12. In a further aspect of the invention, a patient is an adolescent. In certain embodiments, an adolescent subject can be a human subject from ages of about 13 to about 17. In a further aspect of the invention, a patient is an elderly patient. “Elderly” as used herein refers to a patient age 65 years or greater. In a preferred embodiment, the subject is a human.

As used herein the term “effective” applied to dose or amount refers to that quantity of a compound or pharmaceutical composition that is sufficient to result in a desired activity upon administration to a subject in need thereof. By way of example, and not limitation, the desired activity could be the treatment, prevention, and/or improvement of at least one disease, disorder, and/or condition and/or at least one symptom thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

The phrase “pharmaceutically acceptable”, as used in connection with compositions of the invention, refers to molecular entities and other ingredients of such compositions that are physiologically tolerable and do not typically produce untoward reactions when administered to a mammal (e.g., a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans.

As used herein, “amphetamine” means any of the sympathomimetic phenethylamine derivatives which have central nervous system stimulant activity such as, but not limited to, amphetamine (alpha-methyl-phenethylamine), methamphetamine, p-methoxyamphetamine, methylenedioxyamphetamine, 2,5-dimethoxy-4-methylamphetamine, 2,4,5-trimethoxyamphetamine, and 3,4-methylenedioxy-methamphetamine. The amphetamine can be a metabolite of amphetamine, a salt thereof, a derivative thereof, or a mixture thereof. Amphetamine can be in the form of dextro- d-), levo-(1-), or racemic. In an embodiment, the amphetamine is d-amphetamine. An amphetamine-homoarginine conjugate of the present invention includes pharmaceutically acceptable salts of an amphetamine-homoarginine conjugate.

As used herein, “prodrug” means a form of a drug that is not therapeutically active on its own until it is metabolized in the body and made active.

Salts of the homoarginine amphetamine prodrug that can be formed and utilized include, but are not limited to, aspartate, mesylate, hydrochloride, sulfate, oxalate, triflate, citrate, malate, tartrate, phosphate, nitrate, benzoate, acetate, carbonate, hydroxide, sodium, potassium, magnesium, calcium, zinc, saccharate, sulfate, and ammonium salts. Further, in accordance with some embodiments, the salts may be in multiple forms (e.g., di-, tri-, or tetra-). Other derivative forms such as free base, free acid, or neutral forms may also be prepared.

### Indications

The homoarginine amphetamine prodrugs of the presently described technology could be used for any condition requiring the stimulation of the central nervous system (CNS). These conditions include, for example, obesity, narcolepsy, appetite suppressant, depression, anxiety, withdrawals (e.g., alcohol withdrawals or drug withdrawals), and wakefulness. Some stimulants such as amphetamine have also demonstrated usefulness in treating substance (e.g., alcohol, smoking, and drug) abuse and addiction. Amphetamine stimulants have also been used extensively to improve battle field alertness and to combat fatigue.

Chronic fatigue syndrome (CFS), also known as myalgic encephalomyelitis (ME), post-viral fatigue syndrome (PVFS), chronic fatigue immune dysfunction syndrome (CFIDS), and systemic exertion intolerance disease (SEID) is a complicated disorder characterized by extreme fatigue that cannot be explained by an underlying medical condition. The fatigue may worsen with physical or mental activity, but does not improve with rest. Cognitive symptoms (cognitive dysfunctions) in ME/CFS may include difficulty with: memory (including visual memory and verbal memory), attention, information processing, reaction times, and concentration is characterized by extreme physical or mental fatigue not relieved by rest. Patients with ME/CFS can also suffer from inattention.

The terms chronic fatigue syndrome (CFS), myalgic encephalomyelitis (ME), and systemic exertion intolerance disease (SEID) describe medical condition that manifests with a debilitating phenotype of unknown etiology and for which no clear treatment is available. Both diagnosis and treatment of ME/CFS are symptomatic. The 3 core symptoms necessary for diagnosis include disabling fatigue that persists for at least 6 months and results in a greatly diminished ability to perform activities that were easily accomplished before the illness, a worsening of symptoms after physical or mental activity that would not have been problematic before the illness (post-exertional malaise), and unrefreshing sleep. In addition to profound fatigue, sufferers have cognitive dysfunction, autoimmune manifestations, pain not caused by injury, and other symptoms that are worsened upon exertion. Tender lymph nodes, sore throat, digestive issues, orthostatic intolerance, chills and night sweats, and allergies/sensitivities to food, color, chemicals, or noise can also occur.

Fatigue is often a symptom of fibromyalgia. Patients of fibromyalgia often awaken tired, even though they report sleeping for long periods of time. Sleep is often disrupted by pain, and many patients with fibromyalgia have other sleep disorders, such as restless legs syndrome and sleep apnea. Patients with fibromyalgia may also experience cognitive dysfunction and/or inattention.

Narcolepsy is characterized by chronic excessive daytime sleepiness, often with sudden loss of muscle tone (cataplexy). Other symptoms include sleep paralysis and hypnagogic and hypnopompic hallucinations. Diagnosis may be performed, for example, by polysomnography and multiple sleep latency testing. Patients with Narcolepsy may also experience cognitive dysfunction and/or inattention.

Multiple Sclerosis (MS) according to the present invention includes relapsing-remitting, primary progressive, progressive-relapsing, and secondary progressive MS. Fatigue is a common, and often disabling, symptom of MS. Cognitive dysfunctions and/or inattention may also occur in patients with MS.

Major depressive disorder (MDD) according to the present invention may be diagnosed in a patient by a physician according to, for example, the criteria disclosed in the Diagnostic and Statistical Manual of Mental Disorders (DSM-5). Patients with MDD may also experience cognitive dysfunction and/or inattention.

Medications for MDD include selective serotonin reuptake inhibitors (SSRIs) such as fluoxetine, paroxetine, sertraline, citalopram and escitalopram (Lexapro); serotonin-norepinephrine reuptake inhibitors (SNRIs) such as duloxetine, venlafaxine, desvenlafaxine and levomilnacipran; norepinephrine-dopamine reuptake inhibitors (NDRIs) such as bupropion; atypical antidepressants such as trazodone, mirtazapine, vortioxetine and vilazodone; tricyclic antidepressants such as imipramine, nortriptyline, amitriptyline, doxepin, trimipramine, desipramine and protriptyline; and monoamine oxidase inhibitors (MAOIs) such as tranylcypromine, phenelzine and isocarboxazid, and selegiline.

A homoarginine-amphetamine conjugate according to the present invention may be administered as monotherapy for fatigue and/or inattention in a patient suffering from or diagnosed with MDD, or in combination with one or more of the above listed medications for MDD. In one embodiment, the patient suffering from or diagnosed with MDD may be concurrently receiving treatment with a selective serotonin reuptake inhibitors (SSRIs) such as fluoxetine, paroxetine, sertraline, citalopram and escitalopram (Lexapro); serotonin-norepinephrine reuptake inhibitors (SNRIs) such as duloxetine, venlafaxine, desvenlafaxine and levomilnacipran; norepinephrine-dopamine reuptake inhibitors (NDRIs) such as bupropion; atypical antidepressants such as trazodone, mirtazapine, vortioxetine and vilazodone; tricyclic antidepressants such as imipramine, nortriptyline, amitriptyline, doxepin, trimipramine, desipramine and protriptyline; and monoamine oxidase inhibitors (MAOIs) such as tranylcypromine, phenelzine and isocarboxazid, and selegiline.

In an aspect of the invention, a homoarginine-amphetamine conjugate is administered to treat fatigue, cognitive dysfunction, and/or inattention in a patient whose major depressive disorder was inadequately treated by one or more of the above listed medications for MDD. In one embodiment, the patient's MDD may have been inadequately treated with a selective serotonin reuptake inhibitors (SSRIs) such as fluoxetine, paroxetine, sertraline, citalopram and escitalopram (Lexapro); serotonin-norepinephrine reuptake inhibitors (SNRIs) such as duloxetine, venlafaxine, desvenlafaxine and levomilnacipran; norepinephrine-dopamine reuptake inhibitors (NDRIs) such as bupropion; atypical antidepressants such as trazodone, mirtazapine, vortioxetine and vilazodone; tricyclic antidepressants such as imipramine, nortriptyline, amitriptyline, doxepin, trimipramine, desipramine and protriptyline; and monoamine oxidase inhibitors (MAOIs) such as tranylcypromine, phenelzine and isocarboxazid, and selegiline.

In a further aspect of the invention, a homoarginine-amphetamine conjugate of the present invention is administered to a patient with MDD in whom cognition (e.g., attention, fatigue) is not improved with a MDD medication. In one embodiment, the patient's MDD symptoms may have not improved upon treatment with a selective serotonin reuptake inhibitors (SSRIs) such as fluoxetine, paroxetine, sertraline, citalopram and escitalopram (Lexapro); serotonin-norepinephrine reuptake inhibitors (SNRIs) such as duloxetine, venlafaxine, desvenlafaxine and levomilnacipran; norepinephrine-dopamine reuptake inhibitors (NDRIs) such as bupropion; atypical antidepressants such as trazodone, mirtazapine, vortioxetine and vilazodone; tricyclic antidepressants such as imipramine, nortriptyline, amitriptyline, doxepin, trimipramine, desipramine and protriptyline; and monoamine oxidase inhibitors (MAOIs) such as tranylcypromine, phenelzine and isocarboxazid, and selegiline.

In one aspect, the present invention provides a method of treating or preventing cancer associated fatigue, cognitive dysfunction, and/or inattention which comprises administering to a patient in need thereof a therapeutically effective amount of an amphetamine-homoarginine conjugate or a salt thereof.

The terms “cancer” and “cancerous” refer to or describe the physiological condition in mammals that is typically characterized by unregulated cell growth. Examples of cancer include, but are not limited to, carcinoma, lymphoma, blastoma, sarcoma, and leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, melanoma, multiple myeloma and B-cell lymphoma, brain, as well as head and neck cancer, and associated metastases. Additional examples of cancer can be found in The Merck Manual of Diagnosis and Therapy, 19th Edition, § on Hematology and Oncology, published by Merck Sharp & Dohme Corp., 2011 (ISBN 978-0-911910-19-3); The Merck Manual of Diagnosis and Therapy, 20th Edition, § on Hematology and Oncology, published by Merck Sharp & Dohme Corp., 2018 (ISBN 978-0-911-91042-1) (2018 digital online edition at internet website of Merck Manuals); and SEER Program Coding and Staging Manual 2016, each of which are incorporated by reference in their entirety for all purposes.

In one aspect, the present invention provides a method of treating or preventing post-cancer therapy fatigue, cognitive dysfunction, and/or inattention which comprises administering to a patient in need thereof a therapeutically effective amount of an amphetamine-homoarginine conjugate or a salt thereof. In various embodiments of the invention, the cancer therapy is chemotherapy, radiation therapy, surgery, or a combination thereof. In one embodiment, the cancer therapy is a combination of at least two of the following: chemotherapy, radiation therapy, and surgery. In various embodiments, the amphetamine-homoarginine conjugate or a salt thereof is initially administered prior to the cancer therapy, after cancer therapy and before the patient is diagnosed with post-cancer therapy fatigue, cognitive dysfunction, and/or inattention, or after cancer therapy and after the patient is diagnosed with post-cancer therapy fatigue, cognitive dysfunction, and/or inattention.

The cancer therapy may be chemotherapy, radiation therapy, surgery, or any combination of these therapies. In some embodiments, the chemotherapy is methotrexate, cytarabine, vincristine, a steroid (e.g., dexamethasone, hydrocortisone, prednisone) or a combination thereof. According to the present invention, the chemotherapy may have been delivered directly to the central nervous system and/or systemic chemotherapy. In an aspect of the present invention, the radiation therapy is cranial radiation therapy. In a further aspect, the surgery is cranial surgery.

In non-limiting examples, the patient received chemotherapy or radiation therapy for ALL, breast cancer, or a malignant brain cancer. The malignant brain cancer may be, for example, a glioma, astrocytoma, medulloblastoma, ependymoma, or brain stem glioma.

As used herein, “a post-cancer therapy condition” (e.g., post-cancer therapy fatigue or post-cancer therapy inattention) means a condition that occurs after a patient has undergone therapy for a cancer. Cancer therapy includes chemotherapy, radiation therapy (for example, cranial radiation therapy), surgery (for example, cranial surgery), and combinations thereof.

As used herein, “post-cancer therapy” and “post-cancer treatment” mean that a patient has undergone cancer therapy, which may include chemotherapy, radiation therapy (for example, cranial radiation therapy), surgery (for example, cranial surgery), and combinations thereof. Non-limiting examples of chemotherapy include glucocorticoids, methotrexate, 5-fluorouracil, doxorubicin, taxanes (e.g., docetaxel, paclitaxel), cisplatin, cyclophosphamide, capecitabine, and combinations thereof.

Fatigue caused by chemotherapy or radiation therapy may not end even when therapy is complete. Any chemotherapy drug may result in fatigue. To some people fatigue lasts only a couple of days. To others, fatigue persists through and beyond completion of treatment. Drugs such as vincristine, vinblastine, and cisplatin often cause fatigue. Radiation therapy can cause cumulative fatigue (fatigue that increases over time). This can occur regardless of treatment site. Fatigue usually lasts from 3-4 weeks after treatment stops but can continue for up to 2-3 months.

In an aspect of the present invention, the patient suffering from or diagnosed with MDD has primary inattentive symptoms. In a further aspect, the patient suffering from or diagnosed with MDD is elderly.

In addition to the disorders and conditions listed above, the methods for treating fatigue, cognitive dysfunction, and/or inattentiveness comprising administering an amphetamine—homoarginine conjugate according to the present invention are suitable for treating fatigue, cognitive dysfunction, and/or inattentiveness associated with traumatic brain injury, menopause, negative symptoms of schizophrenia, and diabetes (Types I and II).

The symptoms of cognitive dysfunction, which can be used interchangeably with cognitive impairment, can include by way of example and not limitation, an impairment in cognition, including memory [visual memory, verbal memory, short- and long-term memory]), attention (sustained, selective and divided), concentration, comprehension, decision making (logic and reasoning), planning, executive functions, information processing (including auditory, visual, simple, complex, and speed/reaction times, and/or learning.

### Non-Limited Examples of Behavioral Tests

Fatigue may not be relieved by rest. It is a nearly constant state of weariness that develops over time and reduces one's energy, motivation and concentration. Fatigue is a subjective lack of physical or mental energy to carry out usual activities. Fatigue according to the present invention can be mental or physical. Fatigue can be measured, for example, using the Fatigue Severity Scale (FSS), the Modified Fatigue Impact Scale (MFIS), and the Numerical Rating Scale (NRS).

Cognitive dysfunction may be assessed by a cognitive performance instrument. As used herein, the term “cognitive performance instrument” includes a cognitive performance test that can be used to evaluate, classify and/or quantify one or more cognitive, adaptive motor and/or executive functions in a subject. As will be understood by those skilled in the art, such a test may be questionnaire or survey filled out by a patient, caregiver, parent, teacher, therapist or psychologist. Exemplary cognitive performance instruments suitable for assessing cognitive, adaptive motor and/or executive functions are described below.

In some specific embodiments, the cognitive performance instrument is the Differential Ability Scale. The Differential Ability Scale, as the name implies, was developed specifically to be suitable for patients with various types of impairment. The DAS-II is a cognitive test that is designed primarily as a profile test which yields scores for a wide range of abilities, measured either by subtests or composites. However, it has been used as a general test of cognitive ability, including in severely affected populations. The DAS-II has been validated and normed in the US population and in the British population (as the BAS, or British Abilities Scales). A Spanish version, intended for use in Spain and Spanish-speaking Latin America, is expected to become available in the fall of 2012. The DAS-II incorporates “tailored testing” to enable examiners to select the most appropriate items for a child. This has two major advantages. First, it enables the measure to be both accurate and very time-efficient, which is a major advantage for the examiner. Second, it makes testing shorter and less tiring for the child and often enables the child to discontinue a subtest before having experienced a string of consecutive failures—an advantage for the child, as the tests are more enjoyable and motivating. Without being a limiting example, Table 1 discloses a plurality of subtest capable of measuring different cognitive abilities, for a subject undergoing enzyme replacement therapy.

TABLE 1
List of Cognitive Performance Instruments

| Subtest | Abbreviation | Abilities Measured |
|---|---|---|
| Copying | Copy | Visual-perceptual matching |
| | | and fine-motor coordination |
| | | in copying line drawings |
| Early number | ENC | Knowledge of pre-numerical |
| concepts | | and numerical concepts |
| Matching | MLLF | Visual discrimination among |
| letter-like | | similar shapes |
| forms | | |
| Matrices | Mat | Nonverbal reasoning: perception |
| | | and application of relationships |
| | | among abstract figures |
| Naming | NVoc | Expressive language; knowledge |
| vocabulary | | of names |
| Pattern | PCon | Visual-perceptual matching, |
| construction | | especially of spatial orientation, |
| | | in copying block patterns. |
| | | Nonverbal reasoning and spatial |
| | | visualization in reproducing |
| | | designs with colored blocks |
| Pattern | PCon(A) | The same abilities for Pattern |
| Construction | | construction without a time |
| (alt) | | constraint |
| Phonological | PhP | Knowledge of sound structure |
| processing | | of the English language and the |
| | | ability to manipulate sound |
| Picture | PSim | Nonverbal reasoning shown by |
| similarities | | matching pictures that have a |
| | | common element or concept |
| Rapid naming | RNam | Automaticity of integration of |
| | | visual symbols with phonologically |
| | | referenced naming |
| Recall of | RDes | Short-term recall of visual and |
| designs | | spatial relationships through |
| | | reproduction of abstract figures |
| Recall of | DigF | Short-term auditory memory and |
| digits | | oral recall of sequences of numbers |
| forward | | |
| Recall of | DigB | Short-term auditory memory and |
| digits | | oral recall of sequences of numbers |
| backward | | |
| Recall of | RObI | Short-term recall of verbal |
| objects- | | and pictorial information |
| Immediate | | |
| Recall of | RObD | Intermediate-term recall of |
| objects- | | verbal and pictorial information |
| Delayed | | |
| Recall of | SeqO | Short-term recall of verbal and |
| sequential | | pictorial information |
| order | | |
| Recognition | RPic | Short-term, nonverbal visual |
| of pictures | | memory measure through recognition |
| | | of familiar objects |
| Sequential and | SQR | Detection of sequential patterns |
| quantitative | | in figures or numbers |
| reasoning | | |
| Speed of | SIP | Quickness in performing simple |
| information | | mental operations |
| processing | | |
| Verbal | VCom | Receptive language: understanding |
| comprehension | | of oral instructions involving |
| | | basic language concepts |
| Verbal | VSim | Verbal reasoning and verbal knowledge |
| similarities | | |
| Word | WDef | Knowledge of word meanings as |
| definitions | | demonstrated through spoken language |

In some specific embodiments, the cognitive performance instrument is the scales of independent behavior-revised. The Scales of Independent Behavior-Revised (SIB-R) is a measure of adaptive behavior comprising 14 subscales organized into 4 adaptive behavior clusters: (1) Motor skills, (2) Social Interaction/Communication, (3) Personal Living skills and (4) Community and Living skills. For each item, the rater is presented with statements that ask them to evaluate the ability and frequency with which the individual being rated can or does perform, in its entirety, a particular task without help or supervision. The individual's performance is rated on a 4-point Likert scale, with responses including (0): Never or Rarely—even if asked; (1) Does, but not Well—or about one quarter of the time—may need to be asked; (2) does fairly well—or about three quarters of the time—may need to be asked; (3) does very well—always or almost always without being asked.

It also measures 8 areas of problem behavior. The SIB-R provides norms from infancy through to the age of 80 and above. It has been used in children with autism and intellectual disability. Some experts consider that one of the strengths of the SIB-R is that has application for basic adaptive skills and problem behaviors of children with significant cognitive or autistic spectrum disorders and can map to American Association of Mental Retardation levels of support. The SIB-R is considered to be much less vulnerable to exaggeration than some other measures of adaptive behaviors.

In some embodiments, Vineland Adaptive Behavior Scales are performed. Typically, Vineland Adaptive Behavior Scales measure a person's adaptive level of functioning. Typically, the content and scales of Vineland Adaptive Behavior Scales are organized within a three domain structure: Communication, Daily Living, and Socialization. This structure corresponds to the three broad Domains of adaptive functioning recognized by the American Association of Mental Retardation (AAMR, 2002): Conceptual, Practical, and Social. In addition, Vineland Adaptive Behavior Scales offer a Motor Skills Domain and an optional Maladaptive Behavior Index to provide more in-depth information.

Inattention, as used herein, may be measured according to any method known in the art. For example, inattention may be found if six or more of the following symptoms is present for at least six months in a child up to the age of 16, or five or more of the following symptoms is present for at least six months in an adult age 17 or greater:
Often fails to give close attention to details or makes careless mistakes in schoolwork, at work, or with other activities.
Often has trouble holding attention on tasks or play activities.
Often does not seem to listen when spoken to directly.
Often does not follow through on instructions and fails to finish schoolwork, chores, or duties in the workplace (e.g., loses focus, side-tracked).
Often has trouble organizing tasks and activities.
Often avoids, dislikes, or is reluctant to do tasks that require mental effort over a long period of time (such as schoolwork or homework).
Often loses things necessary for tasks and activities (e.g. school materials, pencils, books, tools, wallets, keys, paperwork, eyeglasses, mobile telephones).
Is often easily distracted.
Is often forgetful in daily activities.

Other non-limiting examples of inattentiveness tests include symbol digit modalities (SDMT) and the Connor's Continuous Performance Test (CPT).

A defining clinical feature of CFS is central nervous system (CNS) lesions, which may result in cognitive dysfunction. Additionally, MRI scans of affected subjects can be used to visualize such lesions.

The PROMIS Fatigue SF 7 and PROMIS Cognitive Function Abilities and Concerns item banks and 8-item short forms may be used to assess key patient-reported symptoms of ME/CFS, giving insight into how patients feel and function. In the absence of ME/CFS-specific PRO measures, these PROMIS measures assess highly relevant concepts and offer the rigorous development and validation process of PROMIS measures, which may be supplemented with additional studies in patients from the target population. This methodology is specifically described in Examples 5 and 6.

### Homoarginine-Amphetamine Conjugates

According to the present invention, homoarginine can be chemically attached to amphetamine (d-, 1-, or racemic form or a mixture thereof) to produce homoarginine prodrugs of amphetamine. Amphetamine-homoarginine conjugates and salts of the conjugate, and methods for synthesizing the conjugates, are disclosed in U.S. Pat. Nos. 7,776,917, 7,772,222, and 8,101,661, and U.S. Patent Publication No. 2014/0171510, which are hereby incorporated by reference in their entirety. Metabolites and derivatives of amphetamine can also be so modified. Examples of metabolites of amphetamine include N-hydroxyamphetamine, 4-hydroxyamphetamine, α-hydroxyamphetamine, norephedrine, 4-hydroxynorephedrine, phenylacetone oxime, phenylacetone and 1-phenyl-2-propanol.

In one aspect, the amphetamine-homoarginine conjugate is 1-homoarginine-d-amphetamine dihydrochloride, which is represented by the following Formula I:

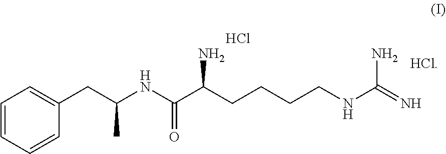

Chemically, the compound of Formula I is a prodrug of d-amphetamine and 1-homoarginine in which the C terminus of 1-homoarginine is covalently bonded via an amide linkage to the primary amine of d-amphetamine. The synthesis is carried out by the coupling of dextroamphetamine with a homoarginine analog to form the amide bond. The homoarginine fragment can be synthesized through various approaches. In one method, L-homoarginine or nitro homoarginine thereof protected at the amine functionality is coupled with dextroamphetamine. The intermediate is deprotected to provide the desired target compound. In another approach, the guanidine functionality is introduced in L-lysine, and the intermediate is reacted with dextroamphetamine, and finally deprotected to furnish the desired compound. Accordingly, the invention also provides methods comprising providing, administering, prescribing, or consuming an amphetamine-homoarginine conjugate salt prodrug. The invention also provides pharmaceutical compositions comprising an amphetamine-homoarginine conjugate salt prodrug. The formulation of such a pharmaceutical composition can optionally enhance or achieve the desired release profile.

In an aspect of the present invention, the amphetamine-homoarginine conjugate salt dose provides the equivalent of about 5 mg to about 40 mg of amphetamine freebase. Alternatively, the dose may also be provided in an equivalent of about 9 mg to about 30 mg of amphetamine freebase. In a further aspect, the conjugates or salts thereof are present in the composition in an amount equivalent to amphetamine freebase in the range of about 5 mg to about 40 mg.

In aspects of the invention, a physician titrates the dosage of an amphetamine-homoarginine conjugate salt (i.e., adjusts the amount and/or dosage frequency) to achieve the desired effect (improvement in inattention) with acceptable or absent adverse effects. For example, a starting dose may be 30 mg once daily. If a dose increase is warranted in the judgment of the physician, the daily dose may be adjusted in increments of 10 mg or 20 mg at approximately weekly intervals.

The dose range for adult or pediatric human patients may depend on a number of factors including the age, weight, and condition of the patient. Suitable oral dosages of the prodrugs of the present invention can be the equivalents of the doses typically found in treatments using that drug. For example, typical dosages for amphetamine salts can range from about 1 mg to about 100 mg. Preferred doses of the prodrug are doses equimolar to amphetamine freebase in the range from about 5 mg to about 40 mg. Preferred doses of the prodrug are doses equimolar to amphetamine freebase in the range from about 9 mg to about 30 mg. For example, doses of a preferred homoarginine amphetamine dichloride prodrug in the range of about 25 mg to about 75 mg would provide an amphetamine freebase content in the preferred range of about 9 mg to about 30 mg. Using the molecular weight of the prodrug of the present technology, the release percentage (% release) of amphetamine from the prodrug and desired dosage forms of the required amphetamine, the following equation can be generated:

grams of a prodrug needed=(dosage/molecular weight of amphetamine)(% release)(molecular weight of the prodrug)

Table 2, below, provides non-limiting illustrative examples of dosage strength conversions for 5.0 mg, 50.0 mg, and 100.0 mg dosages according to an embodiment of the invention.

TABLE 2
Example Dosage Strength Conversions

| | | Conver- | Dosage | | | |
|---|---|---|---|---|---|---|
| Compound | MW | sion | Strengths | | | |
| l-homoarginine-d-am- | 378.34 | 1 | 5.0 mg | 50.0 mg | 100.0 | mg |
| phetamine dihydro- | | | | | | |
| chloride salt | | | | | | |
| l-homoarginine-d-am- | 305.42 | 0.807 | 4.0 mg | 40.4 mg | 80.7 | mg |
| phetamine Free Base | | | | | | |
| D-amphetamine Base | 135.21 | 0.357 | 1.8 mg | 17.9 mg | 35.7 | mg |
| Homoarginine | 188.23 | 0.498 | 2.5 mg | 24.9 mg | 49.8 | mg |

Tablets, capsules, and other forms of unit dosages may conveniently contain a daily dose, or an appropriate fraction thereof, of one or more of the prodrug compounds of the invention. For example, the units may contain from about 1 mg to about 1000 mg, alternatively from about 5 mg to about 500 mg, alternatively from about 5 mg to about 250 mg, alternatively from about 5 mg to about 150 mg, alternatively from about 10 mg to about 100 mg of one or more of the prodrug compounds of the presently described technology. Preferred units of the prodrug are dose units equimolar to amphetamine freebase in the range from about 9 mg to about 27 mg.

In one embodiment, the amphetamine-homoarginine conjugate salt prodrug itself exhibits a sustained release profile. Thus, the invention provides a pharmaceutical composition exhibiting a sustained release profile due to the amphetamine-homoarginine conjugate salt prodrug. For example, the dosage form may be, but is not limited to, an immediate release (IR) form, an IR-delayed form, IR-delayed pulsatile (DPR) form, IR-sustained release (IR-SR) form, IR-DPR-SR form, IR-SR-SR form. Additional non-limiting examples of the dosage forms can be found in U.S. Pat. Nos. 6,322,819; 6,605,300; RE41148; RE42096; U.S. Pat. Nos. 6,913,768 7,105,486; 722,735; 7,662,787; 7,655,630; 7,659,253; 7,659,254; 7,662,787; 7,662,788; 7,671,030; 7,671,031; 7,674,774; 7,678,770; 7,678,771; 7,687,466; 7,687,467; 7,700,561; 7,713,936; 7,718,619; 8,846,100; and 9,173,857, each of which are incorporated by reference herein in their entirety.

Non-limiting examples of dosage forms according to the present invention include chewable tablets, quick dissolve tablets, effervescent tablets, reconstitutable powders, elixirs, liquids, solutions, suspensions, emulsions, tablets, multi-layer tablets, bi-layer tablets, capsules, soft gelatin capsules, hard gelatin capsules, caplets, troches, lozenges, chewable lozenges, beads, powders, granules, particles, microparticles, dispersible granules, cachets, thin strips, oral films, transdermal patches, and combinations thereof.

A dosage form according to the present invention may combine forms of release known to persons of ordinary skill in the art. These conventional release forms include immediate release, extended release, pulse release, variable release, controlled release, timed release, sustained release, delayed release, long acting, and combinations thereof. The ability to combine immediate release, extended release, pulsed release, controlled release, timed release, sustained release, delayed release, and combinations thereof is known in the art.

In another embodiment, a sustained release profile is enhanced or achieved by including a hydrophilic polymer in the pharmaceutical composition. Suitable hydrophilic polymers include, but are not limited to, natural or partially or totally synthetic hydrophilic gums such as acacia, gum tragacanth, locust bean gum, guar gum, and karaya gum; cellulose derivatives such as methyl cellulose, hydroxymethyl cellulose, hydroxypropylmethyl cellulose, hydroxypropyl cellulose, hydroxyethyl cellulose, and carboxymethyl cellulose; proteinaceous substances such as agar, pectin, carrageen, and alginates; hydrophilic polymers such as carboxypolymethylene; gelatin; casein; zein; bentonite; magnesium aluminum silicate; polysaccharides; modified starch derivatives; and other hydrophilic polymers known in the art. Preferably, the hydrophilic polymer forms a gel that dissolves slowly in aqueous acidic media thereby allowing the amphetamine-homoarginine conjugate salt to diffuse from the gel in the stomach. Then when the gel reaches the higher pH medium of the intestines, the hydrophilic polymer dissolves in controlled quantities to allow further sustained release. Preferred hydrophilic polymers are hydroxypropyl methylcelluloses such as Methocel ethers, e.g., Methocel E10M® (Dow Chemical Company, Midland, Mich.). One of ordinary skill in the art would recognize a variety of structures, such as bead constructions and coatings, useful for achieving particular release profiles. See, e.g., U.S. Pat. No. 6,913,768.

In addition to the amphetamine-homoarginine conjugate salt, the pharmaceutical compositions of the invention further comprise one or more pharmaceutical additives. Pharmaceutical additives include a wide range of materials including, but not limited to diluents and bulking substances, binders and adhesives, lubricants, glidants, plasticizers, disintegrants, carrier solvents, buffers, colorants, flavorings, sweeteners, preservatives and stabilizers, and other pharmaceutical additives known in the art. For example, in a preferred embodiment, the pharmaceutical composition comprises magnesium stearate. In another preferred embodiment, the pharmaceutical composition comprises microcrystalline cellulose (e.g., Avicel® PH-102), croscarmellose sodium, and magnesium stearate.

Diluents increase the bulk of a dosage form and may make the dosage form easier to handle. Exemplary diluents include, but are not limited to, lactose, dextrose, saccharose, cellulose, starch, and calcium phosphate for solid dosage forms, e.g., tablets and capsules; olive oil and ethyl oleate for soft capsules; water and vegetable oil for liquid dosage forms, e.g., suspensions and emulsions. Additional suitable diluents include, but are not limited to, sucrose, dextrates, dextrin, maltodextrin, microcrystalline cellulose (e.g., Avicel®), microfine cellulose, powdered cellulose, pregelatinized starch (e.g., Starch 1500®), calcium phosphate dihydrate, soy polysaccharide (e.g., Emcosoy®), gelatin, silicon dioxide, calcium sulfate, calcium carbonate, magnesium carbonate, magnesium oxide, sorbitol, mannitol, kaolin, polymethacrylates (e.g., Eudragit®), potassium chloride, sodium chloride, and talc. A preferred diluent is microcrystalline cellulose (e.g., Avicel® PH-102). Preferred ranges for the amount of diluent by weight percent include about 40% to about 90%, about 50% to about 85%, about 55% to about 80%, about 50% to about 60%, and increments therein.

In embodiments where the pharmaceutical composition is compacted into a solid dosage form, e.g., a tablet, a binder can help the ingredients hold together. Binders include, but are not limited to, sugars such as sucrose, lactose, and glucose; corn syrup; soy polysaccharide, gelatin; povidone (e.g., Kollidon®, Plasdone®); Pullulan; cellulose derivatives such as microcrystalline cellulose, hydroxypropylmethyl cellulose (e.g., Methocel®), hydroxypropyl cellulose (e.g., Klucel®), ethylcellulose, hydroxyethyl cellulose, carboxymethylcellulose sodium, and methylcellulose; acrylic and methacrylic acid co-polymers; carbomer (e.g., Carbopol®); polyvinylpolypyrrolidine, polyethylene glycol (Carbowax®); pharmaceutical glaze; alginates such as alginic acid and sodium alginate; gums such as acacia, guar gum, and arabic gums; tragacanth; dextrin and maltodextrin; milk derivatives such as whey; starches such as pregelatinized starch and starch paste; hydrogenated vegetable oil; and magnesium aluminum silicate.

For tablet dosage forms, the pharmaceutical composition is subjected to pressure from a punch and dye. Among other purposes, a lubricant can help prevent the composition from sticking to the punch and dye surfaces. A lubricant can also be used in the coating of a coated dosage form. Lubricants include, but are not limited to, magnesium stearate, calcium stearate, zinc stearate, powdered stearic acid, glyceryl monostearate, glyceryl palmitostearate, glyceryl behenate, silica, magnesium silicate, colloidal silicon dioxide, titanium dioxide, sodium benzoate, sodium lauryl sulfate, sodium stearyl fumarate, hydrogenated vegetable oil, talc, polyethylene glycol, and mineral oil. A preferred lubricant is magnesium stearate. The amount of lubricant by weight percent is preferably less than about 5%, more preferably 4%, 3%, 2%, 1.5%, 1%, or 0.5%, or increments therein.

Glidants can improve the flowability of non-compacted solid dosage forms and can improve the accuracy of dosing. Glidants include, but are not limited to, colloidal silicon dioxide, fumed silicon dioxide, silica gel, talc, magnesium trisilicate, magnesium or calcium stearate, powdered cellulose, starch, and tribasic calcium phosphate.

Plasticizers include both hydrophobic and hydrophilic plasticizers such as, but not limited to, diethyl phthalate, butyl phthalate, diethyl sebacate, dibutyl sebacate, triethyl citrate, acetyltriethyl citrate, acetyltributyl citrate, cronotic acid, propylene glycol, castor oil, triacetin, polyethylene glycol, propylene glycol, glycerin, and sorbitol. Plasticizers are particularly useful for pharmaceutical compositions containing a polymer and in soft capsules and film-coated tablets. In one embodiment, the plasticizer facilitates the release of the amphetamine-homoarginine conjugate salt from the dosage form.

Disintegrants can increase the dissolution rate of a pharmaceutical composition. Disintegrants include, but are not limited to, alginates such as alginic acid and sodium alginate, carboxymethylcellulose calcium, carboxymethylcellulose sodium (e.g., Ac-Di-Sol®, Primellose®), colloidal silicon dioxide, croscarmellose sodium, crospovidone (e.g., Kollidon®, Polyplasdone®), polyvinylpolypyrrolidine (Plasone-XL®), guar gum, magnesium aluminum silicate, methyl cellulose, microcrystalline cellulose, polacrilin potassium, powdered cellulose, starch, pregelatinized starch, sodium starch glycolate (e.g., Explotab®, Primogel®). Preferred disintegrants include croscarmellose sodium and microcrystalline cellulose (e.g., Avicel® PH-102). Preferred ranges for the amount of disintegrant by weight percent include about 1% to about 10%, about 1% to about 5%, about 2% to about 3%, and increments therein.

In embodiments where the pharmaceutical composition is formulated for a liquid dosage form, the pharmaceutical composition may include one or more solvents. Suitable solvents include, but are not limited to, water; alcohols such as ethanol and isopropyl alcohol; methylene chloride; vegetable oil; polyethylene glycol; propylene glycol; and glycerin.

The pharmaceutical composition can comprise a buffer. Buffers include, but are not limited to, lactic acid, citric acid, acetic acid, sodium lactate, sodium citrate, and sodium acetate.

Any pharmaceutically acceptable colorant can be used to improve appearance or to help identify the pharmaceutical composition. See 21 C.F.R., Part 74. Exemplary colorants include D&C Red No. 28, D&C Yellow No. 10, FD&C Blue No. 1, FD&C Red No. 40, FD&C Green #3, FD&C Yellow No. 6, and edible inks. Preferred colors for gelatin capsules include white, medium orange, and light blue.

Flavorings improve palatability and may be particularly useful for chewable tablet or liquid dosage forms. Flavorings include, but are not limited to maltol, vanillin, ethyl vanillin, menthol, citric acid, fumaric acid, ethyl maltol, and tartaric acid. Sweeteners include, but are not limited to, sorbitol, saccharin, sodium saccharin, sucrose, aspartame, fructose, mannitol, and invert sugar.

The pharmaceutical compositions of the invention can also include one or more preservatives and/or stabilizers to improve storagability. These include, but are not limited to, alcohol, sodium benzoate, butylated hydroxy toluene, butylated hydroxyanisole, and ethylenediamine tetraacetic acid.

Other pharmaceutical additives include gelling agents such as colloidal clays; thickening agents such as gum tragacanth and sodium alginate; wetting agents such as lecithin, polysorbates, and laurylsulphates; humectants; antioxidants such as vitamin E, caronene, and BHT; adsorbents; effervescing agents; emulsifying agents, viscosity enhancing agents; surface active agents such as sodium lauryl sulfate, dioctyl sodium sulfosuccinate, triethanolamine, polyoxyethylene sorbitan, poloxalkol, and quaternary ammonium salts; and other miscellaneous excipients such as lactose, mannitol, glucose, fructose, xylose, galactose, sucrose, maltose, xylitol, sorbitol, chloride, sulfate and phosphate salts of potassium, sodium, and magnesium.

The pharmaceutical compositions can be manufactured according to any method known to those of skill in the art of pharmaceutical manufacture such as, for example, wet granulation, dry granulation, encapsulation, direct compression, slugging, etc. For instance, a pharmaceutical composition can be prepared by mixing the amphetamine-homoarginine conjugate salt with one or more pharmaceutical additives with an aliquot of liquid, preferably water, to form a wet granulation. The wet granulation can be dried to obtain granules. The resulting granulation can be milled, screened, and blended with various pharmaceutical additives such as water-insoluble polymers and additional hydrophilic polymers. In one embodiment, an amphetamine-homoarginine conjugate salt is mixed with a hydrophilic polymer and an aliquot of water, then dried to obtain granules of amphetamine-homoarginine conjugate salt encapsulated by hydrophilic polymer.

After granulation, the pharmaceutical composition is preferably encapsulated, e.g., in a gelatin capsule. The gelatin capsule can contain, for example, kosher gelatin, titanium dioxide, and optional colorants. Alternatively, the pharmaceutical composition can be tableted, e.g., compressed and optionally coated with a protective coating that dissolves or disperses in gastric juices.

The pharmaceutical composition is preferably administered orally. Oral administration permits the maximum release of amphetamine, provides sustained release of amphetamine, and maintains abuse resistance. Preferably, the amphetamine-homoarginine conjugate salt releases the amphetamine over a more extended period of time as compared to administering unbound amphetamine.

Soft gel or soft gelatin capsules may be prepared, for example, by dispersing the formulation in an appropriate vehicle (e.g., vegetable oil) to form a high viscosity mixture. This mixture then is encapsulated with a gelatin based film. The industrial units so formed are then dried to a constant weight.

Chewable tablets can be prepared by mixing the amphetamine-homoarginine conjugate salt with excipients designed to form a relatively soft, flavored tablet dosage form that is intended to be chewed. Conventional tablet machinery and procedures (e.g., direct compression, granulation, and slugging) can be utilized.

Film-coated tablets and sprinklings (e.g., capsules or packers containing beads) can be prepared by coating tablets using techniques such as rotating pan coating methods and air suspension methods to deposit a contiguous film layer on a tablet. Examples of film-coating can be found in Felton et al., Expert Opinion on Drug Delivery. 10 (4): 421-35, which is incorporated by reference in its entirety.

Compressed tablets can be prepared by mixing the amphetamine-homoarginine conjugate salt with excipients that add binding qualities. The mixture can be directly compressed, or it can be granulated and then compressed.

The pharmaceutical compositions of the invention can alternatively be formulated into a liquid dosage form, such as a solution or suspension in an aqueous or non-aqueous liquid. The liquid dosage form can be an emulsion, such as an oil-in-water liquid emulsion or a water-in-oil liquid emulsion. The oils can be administered by adding the purified and sterilized liquids to a prepared enteral formula, which then is placed in the feeding tube of a patient who is unable to swallow.

For oral administration, fine powders or granules containing diluting, dispersing, and/or surface-active agents can be presented in a draught, in water or a syrup, in capsules or sachets in the dry state, in a non-aqueous suspension wherein suspending agents may be included, or in a suspension in water or a syrup. Liquid dispersions for oral administration can be syrups, emulsions, or suspensions. The syrups, emulsions, or suspensions can contain a carrier, for example, a natural gum, agar, sodium alginate, pectin, methylcellulose, carboxymethylcellulose, saccharose, saccharose with glycerol, mannitol, sorbitol, and polyvinyl alcohol.

In one aspect, a composition or unit dosage form according to the invention is formulated for sublingual administration, wherein the unit dosage form is a film including one or more disintegrants (e.g., materials that favor disintegration or fast dissolution by virtue of their solubility in water, such as hydrolyzed starches, sugars, and glycerin, which may play a dual role as a plasticizer and disintegrant) and a plasticizing agent, the film having a first portion including amphetamine-homoarginine conjugate salt, and a second portion including pH neutralizing agent, wherein the unit dosage form includes from 0.5 to 5 mg, from 4 to 10 mg, or from 8 to 20 mg of amphetamine-homoarginine conjugate salt and the pH neutralizing agent is present in an amount sufficient to produce a solution having a pH of between 3.0 and 6.0, preferably between 4.5 and 6.5, (e.g., a pH of between 2.5 and 4.5, 3.0 and 6.0, 3.5 and 6.5, 4.5 and 6.5, or 5.0 and 6.0) when the unit dosage form is placed in unbuffered water at pH 7 (e.g., the pH observed within 5 minutes of placing the unit dosage form in 1, 5, or 10 mL of unbuffered water). The film can include from 1 to 50% (w/w) (e.g., 1±0.75%, 2±1.5%, 3±0.5%, 5±2%, 7.5±2.5%, 10±2%, 14±3%, 18±4%, 22±5%, 25±5%, 30±5%, 35±5%, 40±5%, 45±5%, or 50±5% (w/w)) of the one or more disintegrants. In certain embodiments, the unit dosage form further includes a high molecular weight polymer having a weight average molecular weight of greater than 60 KDa selected from hydroxypropyl cellulose, hydroxypropyl methyl cellulose, hydroxyethyl cellulose, carboxymethyl cellulose, and methyl cellulose. In other embodiments, the unit dosage form further includes a low molecular weight polymer having a weight average molecular weight of from 5 KDa to 50 KDa selected from hydroxypropyl cellulose, hydroxypropyl methyl cellulose, hydroxyethyl cellulose, carboxymethyl cellulose, and methyl cellulose. The pH neutralizing agent can be an organic base (e.g., pyridoxine, meglumine, or any organic base described herein) or an inorganic base (e.g., magnesium hydroxide, sodium bicarbonate, or an inorganic base described herein). Suitable film for oral administration of the compositions according to the invention is disclosed in, e.g., U.S. Pat. No. 8,846,074.

In some embodiments, the compounds or compositions described herein are administered intranasally. As used herein, “nasal delivery-enhancing agents” include agents which enhance the release or solubility (e.g., from a formulation delivery vehicle), diffusion rate, penetration capacity and timing, uptake, residence time, stability, effective half-life, peak or sustained concentration levels, clearance and other desired nasal delivery characteristics (e.g., as measured at the site of delivery, or at a selected target site of activity such as the brain) of the compounds or compositions of the invention. Enhancement of mucosal delivery can thus occur by any of a variety of mechanisms, for example by increasing the diffusion, transport, persistence or stability of the compounds or compositions of the invention, enzyme inhibition, increasing membrane fluidity, modulating the availability or action of calcium and other ions that regulate intracellular or paracellular permeation, solubilizing mucosal membrane components (e.g., lipids), changing non-protein and protein sulfhydryl levels in mucosal tissues, increasing water flux across the mucosal surface, modulating epithelial junctional physiology, reducing the viscosity of mucus overlying the mucosal epithelium, reducing mucociliary clearance rates, increasing nasal blood flow and other mechanisms. Suitable mucosal delivery enhancing agents will be clear to a person skilled in the art of pharmacology and are further described hereafter.

Compositions of the invention can be simple aqueous (e.g., saline) solutions. Alternatively, they can contain various additional ingredients which enhance stability and/or nasal delivery of the compounds of the invention. Such additional ingredients are well known in the art. Non-limiting examples of useful additional ingredients for enhancing nasal delivery include, e.g., (a) aggregation inhibitory agents (e.g., polyethylene glycol, dextran, diethylaminoethyl dextran, and carboxymethyl cellulose), (b) charge modifying agents, (c) pH control agents, (d) degradative enzyme inhibitors (e.g., amastatin and bestatin [see, e.g., O'Hagan et al., Pharm. Res. 1990, 7: 772-776 and WO 05/120551]; (e) mucolytic or mucus clearing agents (e.g., n-acetyl-cysteine, propyl gallate and cysteine methionine dimers, chaotropes [see, e.g., WO 04/093917]), (f) ciliostatic agents; (g) membrane penetration enhancing agents, (h) modulatory agents of epithelial junction physiology, such as nitric oxide (NO) stimulators, chitosan, and chitosan derivatives; (i) vasodilator agents, (j) selective transport-enhancing agents, and (k) stabilizing delivery vehicles, carriers, supports or complex-forming agents. See, e.g., EP 037943, EP 094157, EP 173990, EP 214898, EP 215697, EP 327756, EP 490806, U.S. Pat. No. 4,476,116, U.S. Pat. No. 5,759,565, WO 04/093917 and WO 05/120551.

Non-limiting examples of membrane penetration-enhancing agents useful in the compositions of the invention include, e.g., (i) a surfactant (e.g., Tween 80, Poloxamer 188, polysorbates; see also EP 490806, U.S. Pat. No. 5,759,565, and WO04/093917), (ii) a bile salt or bile salt derivative (e.g., unsaturated cyclic ureas and Transcutol), (iii) a phospholipid or fatty acid additive, mixed micelle, liposome, or carrier, (iv) an alcohol, (v) an enamine, (vi) a nitric oxide donor compound (e.g., S-nitroso-N-acetyl-DL-penicillamine, NOR1, NOR4, which are preferably co-administered with an NO scavenger such as carboxy-PITO or doclofenac sodium), (vii) a long-chain amphipathic molecule (e.g., deacylmethyl sulfoxide, azone, sodium lauryl sulfate, oleic acid) (viii) a small hydrophobic penetration enhancer, (ix) sodium salicylate or a salicylic acid derivative (e.g., acetyl salicylate, choline salicylate, salicylamide, etc.), (x) a glycerol ester of acetoacetic acid, (xi) a cyclodextrin or betacyclodextrin derivative, (xii) a medium-chain fatty acid including mono- and diglycerides (e.g., sodium caprate—extracts of coconut oil, Capmul), (xiii) a chelating agent (e.g., citric acid, salicylates), (xiv) an amino acid or salt thereof (e.g. monoaminocarboxlic acids such as glycine, alanine, phenylalanine, proline, hydroxyproline, etc.; hydroxyamino acids such as serine; acidic amino acids such as aspartic acid, glutamic acid, etc; and basic amino acids such as lysine etc., inclusive of their alkali metal or alkaline earth metal salts), (xv) an N-acetylamino acid or salt thereof, (xvi) an enzyme degradative to a selected membrane component, (xvii) an inhibitor of fatty acid synthesis, (xviii) an inhibitor of cholesterol synthesis, (xix) cationic polymers, or any combination thereof. The membrane penetration-enhancing agent can be also selected from small hydrophilic molecules, including but not limited to, dimethyl sulfoxide (DMSO), dimethylformamide, ethanol, propylene glycol, and the 2-pyrrolidones. Additional membrane penetration enhancers include emulsifiers (e.g. sodium oleyl phosphate, sodium lauryl phosphate, sodium lauryl sulfate, sodium myristyl sulfate, polyoxyethylene alkyl ethers, polyoxyethylene alkyl esters, etc.), caproic acid, lactic acid, malic acid and citric acid and alkali metal salts thereof, pyrrolidonecarboxylic acids, alkylpyrrolidonecarboxylic acid esters, N-alkylpyrrolidones, proline acyl esters, and the like; mixed micelles; glycerol esters of acetoacetic acid (e.g., glyceryl-1,3-diacetoacetate or 1,2-isopropylideneglycerine-3-acetoacetate), and triglycerides (e.g., amylodextrin, Estaram 299, Miglyol 810); cyclodextrins and β-cyclodextrin derivatives (e.g., 2-hydroxypropyl-β-cyclodextrin and heptakis (2,6-di-O-methyl-β-cyclodextrin) which can be optionally conjugated with Peptide and further optionally formulated in an oleaginous base; and N-acetylamino acids (N-acetylalanine, N-acetylphenylalanine, Nacetylserine, N-acetylglycine, N-acetyllysine, N-acetylglutamic acid, N-acetylproline, Nacetylhydroxyproline, etc.) and their salts (alkali metal salts and alkaline earth metal salts), as well as other penetration-promoting agents that are physiologically compatible for intranasal delivery. See, e.g., WO 04/093917, WO 05/120551 and Davis and Ilium (Clin. Pharmacokinet 2003, 42: 1107-1128).

Non-limiting examples of useful absorption enhancers include, e.g., surfactants, glycosides, cyclodextrin and glycols. Non-limiting examples of useful bioadhesive agents include, e.g., carbopol, cellulose agents, starch, dextran, and chitosan.

In various embodiments of the invention, a compound of the invention is combined with one or more of the nasal delivery-enhancing agents recited above. These nasal delivery-enhancing agents may be admixed, alone or together, with the nasal carrier and with the compound of the invention, or otherwise combined therewith in a pharmaceutically acceptable formulation or delivery vehicle. For nasal delivery-enhancing agents to be of value within the invention, it is generally desired that any significant changes in permeability of the mucosa be reversible within a time frame appropriate to the desired duration of drug delivery.

Furthermore, there should be no substantial, cumulative toxicity, nor any permanent deleterious changes induced in the barrier properties of the nasal mucosa with long term use.

In addition to the compound of the invention, the nasal carrier and, optionally, one or more further additives and/or agents, the composition of the invention may further comprise one or more additional therapeutic ingredients (or active substances). These therapeutic ingredients can be any compound that elicits a desired activity or therapeutic or biological response in the subject. Non-limiting examples of useful additional therapeutic ingredients is provided in the Combination Treatments section, below.

The proportion of each further component in the nasal composition of the invention may vary depending on the components used. For example, but without being limiting, the amount of nasal carrier may be in the range of from 0.1 to 99.9% by weight of the total weight or volume of the composition. When present, the amount surfactant may be in the range from about 0.01 to about 10% or higher and preferably about 0.05 to about 1.0% by weight of the total volume or weight of the composition, the amount depending on the specific surfactant used. The amount is generally kept as low as possible since above a certain level no further enhancement of absorption can be achieved and also too high of a surfactant level may cause irritation of the nasal mucosa. The amount of delivery enhancing agents may be at least 0.1%, suitably in the range from about 0.5 to 10% of the total weight of the composition. Where the composition is liquid, the enhancing agent may suitably be present in an amount of from 0.1 to 5% w/v of the total composition. Preserving agents may be present in an amount of from about 0.002 to 0.02% by weight of the total weight or volume of the composition.

The useful delivery volume of the pharmaceutical compositions of the invention is limited by the size of the nasal cavity. Suitable delivery volumes will be clear to a person skilled in the art of pharmacology. Preferably, the total composition quantity administered at each nasal application comprises from about 0.02 to 0.5 ml, preferably about 0.07 to 0.3 ml, typically about 0.09-0.1 ml.

The liquid compositions of the invention may be prepared by bringing into intimate admixture a compound the invention in the liquid carrier optionally together with the further ingredients, additives and/or agents. The solid nasal composition of the invention may be prepared in conventional manner. A compound of the invention may be admixed with the carrier particles, e.g. a polymer base or cellulose product in conventional manner, optionally with further ingredients, additives and/or agents as indicated above e.g. a mucosal delivery enhancing agent or surfactant such as disclosed. A compound of the invention may be in solution e.g. an aqueous or alcoholic solution when being mixed with the carrier particles and the solvent evaporated, e.g. under freeze-drying or spray drying. Such drying may be effected under the conventional conditions. Alternatively, the mixture may be compacted or granulated and then be pulverized and/or sieved. If desired the particles may be coated. In one embodiment of the invention, the nasal composition is prepared by lyophilisation. A homogeneous solution, preferably aqueous, containing a compound of the invention and optionally containing further ingredients, additives and/or agents as discussed above, is prepared and then submitted to lyophilisation in analogy with known lyophilisation procedures, and to subsequent drying. The resulting powder may then be dissolved in a liquid excipient or nasal carrier before administration, e.g. to reconstitute nasal drops, gel or spray. Alternatively, it may be administered as such in the form of lyophilized powder or it may be mixed with further ingredients, additives and/or agents as discussed above. For example, a lyophilized powder comprising a compound of the invention but free of any nasal carrier may be prepared and then admixed with the desired nasal carrier or mixture of nasal carriers.

The present invention encompasses any delivery device that is suitable for nasal administration of the compositions of the invention. Preferably, such means administers a metered dosage of the composition. The composition of the present invention may be packed in any appropriate form or container as long as a means is provided to deliver the composition to the nasal mucosa. Non-limiting examples of useful intranasal delivery devices include, e.g., instillation catheters, droppers, unit-dose containers, squeeze bottles pump sprays, airless and preservative-fee sprays, compressed air nebulizers, metered-dose inhalers, insufflators and pressurized metered dose inhalers.

For administration of a liquid in drop form, compositions of the invention can be placed in a container provided with a conventional dropper/closure device, e.g. comprising a pipette or the like, preferably delivering a substantially fixed volume of composition/drop.

For administration of an aqueous solution as a nasal spray, the aqueous solution may be dispensed in spray form by a variety of methods known to those skilled in the art. For example, such compositions will be put up in an appropriate atomising device, e.g. in a pump-atomiser, or the like. The atomising device will be provided with appropriate means, such as a spray adaptor for delivery of the aqueous spray to the naris. Preferably it will be provided with means ensuring delivery of a substantially fixed volume of composition/actuation (i.e. per spray-unit). Examples of nasal sprays include nasal actuators produced by Ing. Erich Pfeiffer GmbH, Radolfzell, Germany (see U.S. Pat. Nos. 4,511,069, 4,778,810, 5,203,840, 5,860,567, 5,893,484, 6,227,415, and 6,364,166. Additional aerosol delivery forms may include, e.g., compressed air-, jet-, ultrasonic-, and piezoelectric nebulizers.

Alternatively, the spray may be bottled under pressure in an aerosol device. The propellant may be a gas or a liquid (e.g. a fluorinated and/or chlorinated hydrocarbon). The spray composition may be suspended or dissolved in a liquid propellant. Stabilizing and/or suspending agents and/or co-solvents may be present.

A dry powder may be readily dispersed in an inhalation device as described in U.S. Pat. No. 6,514,496 and Garcia-Arieta et al., Biol. Pharm. Bull. 2001; 24: 1411-1416.

If desired a powder or liquid may be filled into a soft or hard capsule or in a single dose device adapted for nasal administration. The powder may be sieved before filled into the capsules such as gelatine capsules. The delivery device may have means to break open the capsule. The powdery nasal composition can be directly used as a powder for a unit dosage form. The contents of the capsule or single dose device may be administered using e.g. an insufflator. Preferably it will be provided with means ensuring dosing of a substantially fixed amount of composition.

In another embodiment, the composition of the invention can be provided as a nasal insert having the compound of the invention dispersed therein. The insert may be retained in the naris, but flushed by the nasal mucus, and may be designed to release the compound of the invention at the same place in the naris. Suitable nasal insert types include nasal plugs, tampons and the like. Further examples of nasal inserts, their characteristics and preparation are described in EP 490806.

The dose range of the amphetamine-homoarginine conjugate salt for humans will depend on a number of factors including the age, weight, and condition of the patient. Tablets and other dosage forms provided in discrete units can contain a daily dose, or an appropriate fraction thereof, of one or more amphetamine-homoarginine conjugate salt. The dosage form can contain a dose of about 2.5 mg to about 500 mg, about 10 mg to about 250 mg, about 10 mg to about 100 mg, about 25 mg to about 75 mg, or increments therein of one or more of the amphetamine-homoarginine conjugate salt. In a preferred embodiment, the dosage form contains 30 mg, 50 mg, or 70 mg of an amphetamine-homoarginine conjugate salt.

The dosage form can utilize any one or any combination of known release profiles including, but not limited to immediate release, extended release, pulse release, variable release, controlled release, timed release, sustained release, delayed release, and long acting.

The pharmaceutical compositions of the invention can be administered in a partial, i.e., fractional dose, one or more times during a 24 hour period. Fractional, single, double, or other multiple doses can be taken simultaneously or at different times during a 24 hour period. The doses can be uneven doses with regard to one another or with regard to the individual components at different administration times. Preferably, a single dose is administered once daily. The dose can be administered in a fed or fasted state.

The dosage units of the pharmaceutical composition can be packaged according to market need, for example, as unit doses, rolls, bulk bottles, blister packs, and so forth. The pharmaceutical package, e.g., blister pack, can further include or be accompanied by indicia allowing individuals to identify the identity of the pharmaceutical composition, the prescribed indication (e.g., ADHD), and/or the time periods (e.g., time of day, day of the week, etc.) for administration. The blister pack or other pharmaceutical package can also include a second pharmaceutical product for combination therapy.

It will be appreciated that the pharmacological activity of the compositions of the invention can be demonstrated using standard pharmacological models that are known in the art. Furthermore, it will be appreciated that the inventive compositions can be incorporated or encapsulated in a suitable polymer matrix or membrane for site-specific delivery, or can be functionalized with specific targeting agents capable of effecting site specific delivery. These techniques, as well as other drug delivery techniques, are well known in the art.

In one embodiment, the prodrugs of the present invention (or pharmaceutically acceptable salts thereof) are used in combination with another antidepressant for adjunctive antidepressant therapy.

The active ingredients can be formulated into a single dosage form, or they can be formulated together or separately among multiple dosage forms. The active ingredients can be administered simultaneously or sequentially in any order.

The prodrug (or pharmaceutical salts thereof) can be administered in combination with an antidepressant to treat depression or a depressive disorder, such as major depressive disorder. The prodrug and antidepressant can be can be administered serially (in any order) or together (simultaneously). The active ingredients can be formulated into a single dosage form, or they can be formulated together or separately among multiple dosage forms.

In the adjunctive antidepressant therapy embodiment, the prodrugs of the present invention, and specifically the preferred prodrug salt (1)-homoarginine-(d)-amphetamine, can be paired with various antidepressants for co-therapy, including, but not limited to, serotonin norepinephrine reuptake inhibitors (SNRIs), selective serotonin reuptake inhibitors (SSRIs), and tertiary amine tricyclic norepinephrine reuptake inhibitors. In yet another adjunctive antidepressant therapy embodiment, a prodrug of the present invention is administered with an atypical antidepressant. Monoamine oxidase inhibitors can also be used in combination with the prodrugs of the present invention for adjunctive antidepressant therapy.

Any feature of the above-describe embodiments can be used in combination with any other feature of the above-described embodiments.

### EXAMPLES

The following examples illustrate specific aspects of the instant description. The examples should not be construed as limiting, as the examples merely provide specific understanding and practice of the embodiments and their various aspects.

### Example 1: Primary Pharmacodynamics In Vitro

In a general side-effect profile panel using a wide range of receptors, transporters, ion channels, enzymes and other biological targets that could directly or indirectly influence dopaminergic or noradrenergic neurotransmission in the brain, initial results indicated some 1-homoarginine-d-amphetamine dihydrochloride activity at the α2-adrenergic receptor. However, after completion of a confirmatory half maximal inhibitory concentration (IC₅₀) assay, it was determined that it is unlikely that 1-homoarginine-d-amphetamine dihydrochloride would cause any side-effects related to any of the common enzymes/receptors.

Further in vitro radioligand binding studies demonstrated that intact 1-homoarginine-d-amphetamine dihydrochloride (up to 10 μM) exhibited no significant activity at the human recombinant norepinephrine or dopamine transporter sites (sites involved in the indirect sympathomimetic activity associated with amphetamine). Therefore, there is no direct interaction by 1-homoarginine-d-amphetamine dihydrochloride with primary targets critical for the pharmacological effects of d-amphetamine.

### Example 2: Primary Pharmacodynamics In Vitro

The pharmacodynamic properties of 1-homoarginine-d-amphetamine dihydrochloride in rats were evaluated and compared to lisdexamfetamine dimesylate and vehicle control after oral (PO), intravenous (IV) and intranasal (IN) administration.

Following PO administration, 1-homoarginine-d-amphetamine dihydrochloride (4.37 mg/kg) induced an increase in motor activity in rats, typical of that following exposure to a stimulant, compared to the vehicle control group. Although activity levels for animals in the l-homoarginine-d-amphetamine dihydrochloride group were in general lower than those for the lisdexamfetamine (4.21 mg/kg) comparator group during the initial three hours of the study, the behavioral response was consistent with corresponding plasma concentrations of d-amphetamine over time.

Following IV administration, 1-homoarginine-d-amphetamine dihydrochloride (5.25 mg/kg) had a stimulant effect in rat, as evidenced by an increase in activity of approximately 15%; however, this effect was less than that caused by lisdexamfetamine (5.05 mg/kg; 60% increase in activity) at a dose that contained 25% more d amphetamine that the lisdexamfetamine dose.

The activity levels of rats after a single IN dose of 1-homoarginine-d-amphetamine dihydrochloride (5.25 mg/kg) was not increased when compared to controls. A similar result was observed for the lisdexamfetamine (5.05 mg/kg) comparator group. This suggests that 1-homoarginine-d-amphetamine dihydrochloride did not induce stimulant effect upon intranasal administration at the given dose concentration.

### Example 3: Pharmacokinetics and Drug Metabolism in Animals

A summary of the pharmacokinetic and drug metabolism studies is provided in Table 4 below.

TABLE 4
Summary of Pharmacokinetic Studies

| | | | |
|---|---|---|---|
| Study Number | Species | Dose Route | Description |

Single Dose Studies

| | | | |
|---|---|---|---|
| KP106-ROPK-001 | Rat | PO | PK |
| KP106-RINPK-002 | Rat | IN | PK |
| KP106-RIVPK-003 | Rat | IV | PK |
| KP106-DOPK-000 | Dog | PO | PK |
| KP106-DIVPK-010 | Dog | IV | PK |
| KP106-ROMET-017 | Rat | PO | ADME |
| KP106-ROPK-018 | Rat | PO | Dose Ranging |
| | Rat | PO/IV | PK (LHDA HCl) |
| | Dog | PO/IV | PK (LHDA HCl) |

Repeat Dose Studies

| | | | |
|---|---|---|---|
| KP106-R14T-014 | Rat | PO | 14 Day TK |
| KP106-D28T-015 | Dog | PO | 28 Day TK |
| Planned | In vitro | In vitro | Metabolism - |
| | | | Hydrolysis |
| | | | mechanism |

ADME = absorption, distribution, metabolism, excretion; PK = pharmacokinetic; PO = oral; TK = toxicokinetic.
LHDA is l-homoarginine-d-amphetamine; LHDA HCl is l-homoarginine-d-amphetamine dihydrochloride

### Example 4: Phase 1, Open-Label, Single-Center, Single-Dose, 5-Period, Dose Escalation Study Of L-Homoarginine-D-Amphetamine Dihydrochloride In Healthy Adult Subjects

While this study refers to 1-homoarginine-d-amphetamine dihydrochloride, the same can be conducted with any amphetamine-homoarginine conjugates or salts thereof.

The proposed phase 1 study of 1-homoarginine-d-amphetamine dihydrochloride will be designed to characterize the pharmacokinetics and to assess the safety and tolerability of ascending single-doses of 1-homoarginine-d-amphetamine dihydrochloride in healthy adult subjects. Four single-dose escalation periods will be used to evaluate the pharmacokinetic parameters of 1-homoarginine-d-amphetamine dihydrochloride doses from 20 to 150 mg. A preliminary food effect assessment will occur at one of the previously completed dose levels in period 5. The primary objective will be to characterize the single-dose pharmacokinetic parameters of d-amphetamine in the 1-homoarginine-d-amphetamine dihydrochloride dose range 20 to 150 mg. Secondary objectives will be to characterize the single-dose pharmacokinetic parameters of 1-homoarginine-d-amphetamine dihydrochloride and homoarginine, assess the safety and tolerability, and to determine the dose proportionality of 1-homoarginine-d-amphetamine dihydrochloride over the dose range 20 to 150 mg as well as to assess the preliminary food effect of 1-homoarginine-d-amphetamine dihydrochloride at a previously well-tolerated dose level. In addition, plasma concentrations of 1-homoarginine-d-amphetamine dihydrochloride, 1-homoarginine, and d-amphetamine will be measured using validated bioanalytical assays.

### Example 5: Fatigue and Cognitive Function Assessment

Based on literature reviews of the symptoms and impacts of myalgic encephalomyelitis/chronic fatigue syndrome (ME/CFS) and the clinical outcomes assessments used to measure symptoms of ME/CFS, the PROMIS Fatigue SF 7 and PROMIS Cognitive Function Abilities and Concerns assessments have been identified as measures of patient-reported symptoms, giving insight into how patients with ME/CFS feel and function. These PROMIS measures assess highly relevant concepts and offer rigorous development and validation process, which will be used in combination with additional studies in ME/CFS patients.

PROMIS Fatigue SF 7

The planned primary endpoint measure is the 7-item PROMIS (Patient-Reported Outcomes Measurement Information System) Fatigue Short Form (PROMIS Fatigue SF 7), which assesses fatigue-related symptoms (tiredness, exhaustion, mental tiredness, and lack of energy) and impacts of fatigue on daily activities (activity limitations related to work, self-care, and exercise). Fatigue in ME/CFS is the hallmark symptom of ME/CFS and may be extremely severe and persistent regardless of rest or sleep (Bested and Marshall, 2015). Therefore, a patient-reported assessment of fatigue was chosen as the primary endpoint measure of amphetamine-homoarginine conjugate or salt thereof clinical trials in ME/CFS.

The PROMIS Fatigue instruments “evaluate a range of self-reported symptoms, from mild subjective feelings of tiredness to an overwhelming, debilitating, and sustained sense of exhaustion that likely decreases one's ability to execute daily activities and function normally in family or social roles” (NIH PROMIS, 2014). PROMIS Fatigue items were compiled from existing fatigue measures, and domain experts and patients with chronic conditions provided input during development of the measures (Christodoulou et al., 2008).

The planned primary endpoint is improvement in fatigue-related symptoms and impacts as assessed by the PROMIS Fatigue SF 7. Minimal important difference in the PROMIS Fatigue SF 7 score has been previously estimated as a change of 3 to 5 points in patients with cancer-related fatigue using anchor-based and distribution-based methods (Yost et al., 2011).

Additional qualitative and quantitative analyses of the PROMIS Fatigue SF 7 in studies of patients with ME/CFS will be conducted to evaluate the content validity and other psychometric properties of this PRO measure in the target patient population, including developing a responder definition.

PROMIS Cognitive Function Abilities and Concerns

Planned secondary endpoints in studies of amphetamine-homoarginine conjugates or salts thereof are improvements in patient-reported cognition-related symptoms and impacts as assessed by PROMIS Cognitive Function Abilities and Concerns measures, either the two existing SF 8 measures or two ME/CFS-specific cognitive function scales to be developed. The PROMIS Cognitive Function Abilities items are positive statements related to cognitive functioning such as “My memory has been as good as usual” and “I have been able to concentrate.” Cognitive Function Concerns items address many of the same concepts but are worded negatively (e.g., “My thinking has been slow” and “I have had trouble shifting back and forth between different activities that require thinking.”) PROMIS developers conducted confirmatory factor analysis of the Concerns and Abilities item banks and recommended measuring and reporting Concerns and Abilities separately (Lai et al., 2014).

Patient-reported cognitive function has been selected for assessment because of the prevalence, severity, and impact of cognitive dysfunction reported by patients with ME/CFS. A review of cognitive impairment in ME/CFS (Shanks et al., 2013) notes that research has highlighted the negative impact of ME/CFS on memory (visual memory, verbal memory, short- and long-term memory), concentration, attention, and simple and complex information processing. Problems with cognitive function have been reported by 85% to 95% of patients with ME/CFS (Komaroff and Buchwald, 1991; Grafman, 1993). For the majority of these patients, cognitive problems significantly interfere with daily functioning, particularly work and school functioning (Shanks et al., 2013).

Input from expert clinicians/key opinion leaders with extensive experience treating patients with ME/CFS is being collected to confirm the manifestation of key symptoms and impacts of ME/CFS and the potential relevance and importance of the PROMIS Fatigue and Cognitive Function items for these patients from the clinician viewpoint. Five interviews with physicians specializing in caring for patients with ME/CFS are planned.

Three rounds of qualitative interviews with 10 ME/CFS patients each will be conducted, for a total of 30 patients. The objectives of these interviews include concept elicitation and cognitive debriefing of fatigue measures and cognitive function assessment options.

### Example 6: Methods for Preliminary Psychometric Evaluation of PROMIS Measures of Fatigue and Cognitive Function

Measurement properties of the selected PROMIS fatigue and cognitive function measures will be evaluated. Specifically, a psychometric evaluation will be conducted to confirm the structure, internal consistency reliability and construct validity of the measures, and evaluate longitudinal measurement properties (i.e., test-retest reliability and responsiveness). In addition, responder definitions will be developed to evaluate meaningful change using anchor- and distribution-based methods, as well as cumulative distribution function plots.

Planned psychometric analyses to be conducted at the conclusion of the study will include analyses such as those presented in Table 5. Analyses will include pre-baseline (2 weeks prior), Baseline, Week 12, and Week 24 (end of treatment) data. The psychometric analysis plan will explicitly state hypotheses and subgroups of interest.

TABLE 5
Proposed Analyses for the Psychometric Evaluation
of PROMIS Fatigue and Cognitive Function Measures

| | | |
|---|---|---|
| Psychometric | | |
| Evaluation | Measures Used | Evaluation Method |

Descriptive Statistics

| | | |
|---|---|---|
| Summary | PROMIS Fatigue SF 7 and PROMIS | Mean, SD, median, |
| measures | Cognitive Function Abilities and | frequency distribution, |
| | Concerns scores at Week -2, | percentage missing |
| | Baseline. Week 12, Week 24 | |

Reliability

| | | |
|---|---|---|
| Internal | PROMIS Fatigue SF 7 and PROMIS | Cronbach's coefficient |
| consistency | Cognitive Function Abilities and | alpha |
| reliability | Concerns scores at Baseline, Week | |
| | 12, Week 24 | |
| Test-retest | PROMIS Fatigue SF 7 and PROMIS | ICC |
| | Cognitive Function Abilities and | |
| | Concerns scores at Week -2 and | |
| | Baseline | |

Structure

| | | |
|---|---|---|
| Psychometric | Measures Used | Evaluation Method |
| Evaluation | | |
| | PROMIS Fatigue SF 7 and PROMIS | Inter-item correlations^{a} |
| | Cognitive Function Abilities and | and confirmatory factor |
| | Concerns scores at Baseline, Week | analysis |
| | 12 and Week 24 | |

Validity

| | | |
|---|---|---|
| Construct | Supporting measures^{c} and PROMIS | Correlation^{a} |
| | Fatigue SF 7 and PROMIS Cognitive | |
| | Function Abilities and Concerns | |
| | scores at Baseline, Week 12, and | |
| | Week 24 | |
| Known groups | PGI-S and PROMIS Fatigue SF 7 and | ANOVA^{b} |
| | PROMIS Cognitive Function Abilities | |
| | and Concerns scores at Baseline, Week | |
| | 12, and Week 24 | |

Responsiveness

| | |
|---|---|
| Change in supporting measures^{c} and | Correlation^{a} |
| Change in PROMIS Fatigue SF 7 and | |
| PROMIS Cognitive Function Abilities | |
| and Concerns scores from Baseline | |
| (to Week 12, Week 24) | |
| PROMIS Fatigue SF 7 and PROMIS | Effect size of change |
| Cognitive Function Abilities and | |
| Concerns scores (Change in scores | |
| from Baseline (to Week 12, Week 24)) | |

Interpretation of Change Scores

| | | |
|---|---|---|
| Anchor | PGI-C (much improved) and Change in | Mean change method |
| | PROMIS Fatigue SF 7 and PROMIS | |
| | Cognitive Function Abilities and | |
| | Concerns scores from Baseline (to Week | |
| | 12 and Week 24) | |
| | Change in PGI-S (1-point improvement) | Mean change method |
| | and Change in PROMIS Fatigue SF 7 | Cumulative distribution |
| | and PROMIS Cognitive Function Abilities | plots |
| | and Concerns scores from Baseline | |
| | (to Week 12 and Week 24) | |
| ½ SD at | PROMIS Fatigue SF 7 and PROMIS | Distribution-based |
| baseline | Cognitive Function Abilities and | |
| | Concerns scores at Baseline | |

ANOVA = analysis of variance; ICC = intra-class correlation; PGI-C = Patient Global Impression of Change; PGI-S = Patient Global Impression of Severity; SD = standard deviation.
^{a}Pearson or polyserial correlations
^{b}Between-group comparisons tested
^{c}Supporting measures may include the PGI-S, PGI-C, and PRO measures of fatigue and cognitive function.

As various changes can be made in the above-described subject matter without departing from the scope and spirit of the present invention, it is intended that all subject matter contained in the above description, or defined in the appended claims, be interpreted as descriptive and illustrative of the present invention. Many modifications and variations of the present invention are possible in light of the above teachings. Accordingly, the present description is intended to embrace all such alternatives, modifications, and variances which fall within the scope of the appended claims.

All patents, applications, publications, test methods, literature, and other materials cited herein are hereby incorporated by reference in their entirety as if physically present in this specification.

### REFERENCES

1. Bested A C, Marshall L M. Review of myalgic encephalomyelitis/chronic fatigue syndrome: an evidence-based approach to diagnosis and management by clinicians. Rev Environ Health. 2015;30(4):223-49.

2. Christodoulou C, Junghaenel D U, DeWalt D A, Rothrock N, Stone A A. Cognitive interviewing in the evaluation of fatigue items: results from the patient-reported outcomes measurement information system (PROMIS). Qual Life Res. 2008 December;17(10):1239-46.

3. Food and Drug Administration (FDA). Guidance for industry: patient-reported outcome measures: use in medical product development to support labeling claims. October 2009. Available at: http://www.fda.gov/downloads/Drugs/GuidanceComplianceRegulatoryInformation/Guidances/UCM193282.pdf.

4. Grafman J., Schwartz V., Dale J. K., Scheffers M., Houser C., Straus S. E. (1993).

Analysis of neuropsychological functioning in patients with chronic fatigue syndrome. J. Neurol. Neurosurg. Psychiatry 56, 684-689.

5. Komaroff A L, Buchwald D. Symptoms and signs of chronic fatigue syndrome. Rev Infect Dis. 1991 January-February;13 Suppl 1:S8-11.

6. Lai J S, Wagner L I, Jacobsen P B, Cella D. Self-reported cognitive concerns and abilities: two sides of one coin? Psychooncology. 2014 October;23(10):1133-41.

7. National Institutes of Health (NIH) Patient-Reported Outcome Measurement

Information System (PROMIS). Fatigue: a brief guide to the PROMIS Fatigue instruments. September 2015. Available at: https://www.assessmentcenter.net/documents/PROMIS%20Fatigue%20Scoring%20Manual.pdf.

8. Shanks L, Jason L A, Evans M, Brown A. Cognitive impairments associated with CFS and POTS. Front Physiol. 2013 May 16;4:113.

9. Yost K J, Eton D T, Garcia S F, Cella D. Minimally important differences were estimated for six Patient-Reported Outcomes Measurement Information System-Cancer scales in advanced-stage cancer patients. J Clin Epidemiol 2011 May;64(5):507-16.

10. Atzler D, Schonhoff M, Cordts K, Ortland I, Hoppe J, Hummel F et al. Oral Supplementation with L-Homoarginine in Young Volunteers. British Journal of Pharmacology 2016, 82; 1455-1485.

11. Cox, R. H., et al. Interactions of Caffeine with Various Amphetamines on Rat Food Consumption and Avoidance Responding. Neuropharmacology 1976, 15,767-771.

12. Davis W M, Braude, M. C., et al. Acute Toxicity and Gross Behavioral Effects of Amphetamine, Four Methoxyamphetamines, and Mescaline in Rodents, Dogs and Monkeys. Toxicology and App Pharmacology 1978, 45, 49-62.

13. Everds N E, Snyder P W, Bailey K L, Bolon B, Creasy D M, Foley G L et al. Interpreting Stress Responses During Routine Toxicity Studies: A Review of the Biology, Impact and Assessment. Toxicologic Pathology 2014; 41: 560-614

14. Dantzig A H, Hoskins J A, Tabas L B, Bright S, Shepard R L, Jenkins I L, et al. Association of intestinal peptide transport with a protein related to the cadherin superfamily. Science. 1994;264(5157):430-433.

15. Heal D J (Inventor). Methods, compositions and uses thereof International Application Published Under the Patent Cooperation Treaty (PCT), International Publication Number: WO20081119978 A1. 9 Oct., 2008

16. Heal D J, Smith S L, Kulkarni R S, Rowley H L. New perspectives from microdialysis studies in freely moving, spontaneuously hypertensive rats on the pharmacology of drugs for the treatment of ADHD. Pharmacology Biochemistry Behavior. 2008;90:184 197.

17. Heal D, Gosden J, Smith S. The 5-HT6 receptor as a target for developing novel antiobesity drugs. International Review Neurobiology. 2011;96:73-109.

18. Moncrief, S., Toxicity Profile of Lisdexamfetamine Dimesylate in Three Independent Rat Toxicology Studies. Basic and Clin Pharm and Tox 2007, 101, 231-240.

19. National Toxicology Program. Toxicology and carcinogenesis studies of dl-amphetamine sulfate (CAS 60-13-9) in F344/N Rats and B6C3F1 Mice (Feed Studies). Natl Toxicol Program Tech Rep Ser 1991,387, 1-189.

20. Samanin, R. et al., Role of Brain Monoamines in the Anorectic Activity of Mazindol and d-amphetamine in the Rat. Eur Journ of Pharm 1977, 43, 117-124.

21. Valtonen, P., et al. Serum L-Homoarginine Concentration is Elevated During Normal Pregnancy and is Related to Flow-Mediated Vasodilatation. Circ J 2008; 72, 1879-1884

What is claimed is:

1. A method of treating fatigue in a patient which comprises administering a therapeutically effective amount of an amphetamine-homoarginine conjugate or a salt of the amphetamine-homoarginine conjugate to a patient in need thereof.

2. The method of claim 1, wherein the fatigue is in a patient diagnosed with chronic fatigue syndrome.

3. The method of claim 1, wherein the fatigue is in a patient diagnosed with myalgic encephalomyelitis.

4. The method of claim 1, wherein the fatigue is in a patient diagnosed with fibromyalgia.

5. The method of claim 1, wherein the fatigue is in a patient diagnosed with multiple sclerosis.

6. The method of claim 1, wherein the fatigue is in a patient diagnosed with major depressive disorder.

7. The method of claim 1, wherein the fatigue is in a patient diagnosed with a metabolic disorder.

8. The method of claim 7, wherein the metabolic disorder is obesity, appetite related symptoms of the Prader Willi Syndrome, Type 1 diabetes mellitus, Type 2 diabetes mellitus, or a combination thereof.

9. The method of claim 1, wherein the fatigue is post-cancer therapy fatigue caused by cancer therapy, wherein the cancer therapy is chemotherapy, radiation therapy, surgery, or combinations thereof.

10. The method of claim 9, wherein the chemotherapy is selected from the group consisting of methotrexate, cytarabine, vincristine, dexamethasone, hydrocortisone, and prednisone.

11. The method of claim 9, wherein the chemotherapy is for acute lymphoblastic leukemia, breast cancer, or a malignant brain cancer.

12. The method of claim 11, wherein the malignant brain cancer is a glioma, astrocytoma, medulloblastoma, ependymoma, or brain stem glioma.

13. A method of treating fatigue in a patient diagnosed with chronic fatigue syndrome, which comprises administering a therapeutically effective amount of an amphetamine-homoarginine conjugate or a salt of the amphetamine-homoarginine conjugate to a patient in need thereof.

14. A method of treating fatigue in a patient diagnosed with myalgic encephalomyelitis, which comprises administering a therapeutically effective amount of an amphetamine-homoarginine conjugate or a salt of the amphetamine-homoarginine conjugate to a patient in need thereof.

15. A method of treating fatigue in a patient suffering from fibromyalgia, which comprises administering a therapeutically effective amount of an amphetamine-homoarginine conjugate or a salt of the amphetamine-homoarginine conjugate to a patient in need thereof.

16. A method of treating fatigue in a patient suffering from multiple sclerosis which comprises administering a therapeutically effective amount of an amphetamine-homoarginine conjugate or a salt of the amphetamine-homoarginine conjugate to a patient in need thereof.

17. A method of treating fatigue in a patient suffering from major depressive disorder which comprises administering a therapeutically effective amount of an amphetamine-homoarginine conjugate or a salt of the amphetamine-homoarginine conjugate to a patient in need thereof.

18. The method of claim 17, wherein the patient is receiving concurrent treatment with a selective serotonin reuptake inhibitor, a serotonin norepinephrine reuptake inhibitor, a norepinephrine dopamine reuptake inhibitor, an atypical antidepressant, a tricyclic antidepressant, and/or a monoamine oxidase inhibitor.

19. The method of claim 17 or 18, wherein the patient's fatigue was inadequately treated with a selective serotonin reuptake inhibitor, a serotonin norepinephrine reuptake inhibitor, a norepinephrine dopamine reuptake inhibitor, an atypical antidepressant, a tricyclic antidepressant, and/or a monoamine oxidase inhibitor.

20. A method of treating fatigue in a cancer patient caused by chemotherapy, radiation therapy, surgery, or a combination thereof, which comprises administering a therapeutically effective amount of an amphetamine-homoarginine conjugate or a salt of the amphetamine-homoarginine conjugate to a patient in need thereof.

21. The method of any of claims 1-20, further comprising treating cognitive dysfunction.

22. The method of any of claims 1-21, further comprising treating inattention.

23. A method of treating cognitive dysfunction which comprises administering a therapeutically effective amount of an amphetamine-homoarginine conjugate or a salt of the amphetamine-homoarginine conjugate to a patient in need thereof.

24. The method of claim 23, wherein the cognitive dysfunction is in a patient diagnosed with chronic fatigue syndrome.

25. The method of claim 23, wherein the cognitive dysfunction is in a patient diagnosed with myalgic encephalomyelitis.

26. The method of claim 23, wherein the cognitive dysfunction is in a patient diagnosed with fibromyalgia.

27. The method of claim 23, wherein the cognitive dysfunction is in a patient diagnosed with multiple sclerosis.

28. The method of claim 23, wherein the cognitive dysfunction is in a patient diagnosed with major depressive disorder.

29. The method of claim 23, wherein the cognitive dysfunction is in a patient diagnosed with a metabolic disorder.

30. The method of claim 29, wherein the metabolic disorder is obesity, appetite related symptoms of the Prader Willi Syndrome, Type 1 diabetes mellitus, Type 2 diabetes mellitus, or a combination thereof.

31. The method of claim 23, wherein the cognitive dysfunction is post-cancer therapy cognitive dysfunction caused by cancer therapy, wherein the cancer therapy is chemotherapy, radiation therapy, surgery, or combinations thereof.

32. The method of claim 31, wherein the chemotherapy is selected from the group consisting of methotrexate, cytarabine, vincristine, dexamethasone, hydrocortisone, and prednisone.

33. The method of claim 31, wherein the chemotherapy is for acute lymphoblastic leukemia, breast cancer, or a malignant brain cancer.

34. The method of claim 33, wherein the malignant brain cancer is a glioma, astrocytoma, medulloblastoma, ependymoma, or brain stem glioma.

35. A method of treating cognitive dysfunction in a patient diagnosed with chronic fatigue syndrome, which comprises administering a therapeutically effective amount of an amphetamine-homoarginine conjugate or a salt of the amphetamine-homoarginine conjugate to a patient in need thereof.

36. A method of treating cognitive dysfunction in a patient diagnosed with myalgic encephalomyelitis, which comprises administering a therapeutically effective amount of an amphetamine-homoarginine conjugate or a salt of the amphetamine-homoarginine conjugate to a patient in need thereof.

37. A method of treating cognitive dysfunction in a patient suffering from fibromyalgia, which comprises administering a therapeutically effective amount of an amphetamine-homoarginine conjugate or a salt of the amphetamine-homoarginine conjugate to a patient in need thereof.

38. A method of treating cognitive dysfunction in a patient suffering from multiple sclerosis which comprises administering a therapeutically effective amount of an amphetamine-homoarginine conjugate or a salt of the amphetamine-homoarginine conjugate to a patient in need thereof.

39. A method of treating cognitive dysfunction in a patient suffering from major depressive disorder which comprises administering a therapeutically effective amount of an amphetamine-homoarginine conjugate or a salt of the amphetamine-homoarginine conjugate to a patient in need thereof.

40. The method of claim 39, wherein the patient is receiving concurrent treatment with a selective serotonin reuptake inhibitor, a serotonin norepinephrine reuptake inhibitor, a norepinephrine dopamine reuptake inhibitor, an atypical antidepressant, a tricyclic antidepressant, and/or a monoamine oxidase inhibitor.

41. The method of claim 39 or 40, wherein the patient's cognitive dysfunction was inadequately treated with a selective serotonin reuptake inhibitor, a serotonin norepinephrine reuptake inhibitor, a norepinephrine dopamine reuptake inhibitor, an atypical antidepressant, a tricyclic antidepressant, and/or a monoamine oxidase inhibitor.

42. A method of treating cognitive dysfunction in a cancer patient caused by chemotherapy, radiation therapy, surgery, or a combination thereof, which comprises administering a therapeutically effective amount of an amphetamine-homoarginine conjugate or a salt of the amphetamine-homoarginine conjugate to a patient in need thereof.

43. The method of any of claims 23-42, further comprising treating fatigue.

44. The method of any of claims 23-43, further comprising treating inattention.

45. The method of any of claims 23-44, wherein the cognitive dysfunction is a short-term memory problem, an attention problem, a concentration problem, a planning problem, or a combination thereof.

46. A method of treating inattention which comprises administering a therapeutically effective amount of an amphetamine-homoarginine conjugate or a salt of the amphetamine-homoarginine conjugate to a patient in need thereof.

47. The method of claim 46, wherein the inattention is in a patient diagnosed with chronic fatigue syndrome.

48. The method of claim 46, wherein the inattention is in a patient diagnosed with myalgic encephalomyelitis.

49. The method of claim 46, wherein the inattention is in a patient diagnosed with fibromyalgia.

50. The method of claim 46, wherein the inattention is in a patient diagnosed with multiple sclerosis.

51. The method of claim 46, wherein the inattention is in a patient diagnosed with major depressive disorder.

52. The method of claim 46, wherein the inattention is in a patient diagnosed with a metabolic disorder.

53. The method of claim 52, wherein the metabolic disorder is obesity, appetite related symptoms of the Prader Willi Syndrome, Type 1 diabetes mellitus, Type 2 diabetes mellitus, or a combination thereof.

54. The method of claim 46, wherein the inattention is post-cancer therapy inattention caused by cancer therapy, wherein the cancer therapy is chemotherapy, radiation therapy, surgery, or combinations thereof.

55. The method of claim 54, wherein the chemotherapy is selected from the group consisting of methotrexate, cytarabine, vincristine, dexamethasone, hydrocortisone, and prednisone.

56. The method of claim 45, wherein the chemotherapy is for acute lymphoblastic leukemia, breast cancer, or a malignant brain cancer.

57. The method of claim 56, wherein the malignant brain cancer is a glioma, astrocytoma, medulloblastoma, ependymoma, or brain stem glioma.

58. A method of treating inattention in a patient diagnosed with chronic fatigue syndrome, which comprises administering a therapeutically effective amount of an amphetamine-homoarginine conjugate or a salt of the amphetamine-homoarginine conjugate to a patient in need thereof.

59. A method of treating inattention in a patient diagnosed with myalgic encephalomyelitis, which comprises administering a therapeutically effective amount of an amphetamine-homoarginine conjugate or a salt of the amphetamine-homoarginine conjugate to a patient in need thereof.

60. A method of treating inattention in a patient suffering from fibromyalgia, which comprises administering a therapeutically effective amount of an amphetamine-homoarginine conjugate or a salt of the amphetamine-homoarginine conjugate to a patient in need thereof.

61. A method of treating inattention in a patient suffering from multiple sclerosis which comprises administering a therapeutically effective amount of an amphetamine-homoarginine conjugate or a salt of the amphetamine-homoarginine conjugate to a patient in need thereof.

62. A method of treating inattention in a patient suffering from major depressive disorder which comprises administering a therapeutically effective amount of an amphetamine-homoarginine conjugate or a salt of the amphetamine-homoarginine conjugate to a patient in need thereof.

63. The method of claim 62, wherein the patient is receiving concurrent treatment with a selective serotonin reuptake inhibitor, a serotonin norepinephrine reuptake inhibitor, a norepinephrine dopamine reuptake inhibitor, an atypical antidepressant, a tricyclic antidepressant, and/or a monoamine oxidase inhibitor.

64. The method of claim 62 or 63, wherein the patient's inattention was inadequately treated with a selective serotonin reuptake inhibitor, a serotonin norepinephrine reuptake inhibitor, a norepinephrine dopamine reuptake inhibitor, an atypical antidepressant, a tricyclic antidepressant, and/or a monoamine oxidase inhibitor.

65. A method of treating inattention in a cancer patient caused by chemotherapy, radiation therapy, surgery, or a combination thereof, which comprises administering a therapeutically effective amount of an amphetamine-homoarginine conjugate or a salt of the amphetamine-homoarginine conjugate to a patient in need thereof.

66. The method of any of claims 46-65, further comprising treating cognitive dysfunction.

67. The method of any of claims 46-66, further comprising treating fatigue.

68. The method of any of claim 9-12, 20-22, 31-34, 42-45, 54-57, or 65-67 wherein the amphetamine-homoarginine conjugate or a salt of the amphetamine-homoarginine conjugate is initially administered prior to the cancer therapy.

69. The method of any of claim 9-12, 20-22, 31-34, 42-45, 54-57, or 65-67, wherein the amphetamine-homoarginine conjugate or a salt of the amphetamine-homoarginine conjugate is initially administered after cancer therapy and before the patient is diagnosed with fatigue caused by chemotherapy or radiation therapy.

70. The method of any of claim 9-12, 20-22, 31-34, 42-45, 54-57, or 65-67, wherein the amphetamine-homoarginine conjugate or a salt of the amphetamine-homoarginine conjugate is initially administered after cancer therapy and after the patient is diagnosed with fatigue caused by chemotherapy or radiation therapy.

71. The method of any of claim 9-12, 20-22, 31-34, 42-45, 54-57, or 65-67, wherein the patient received chemotherapy that was delivered directly to the central nervous system.

72. The method of any of claim 9-12, 20-22, 31-34, 42-45, 54-57, or 65-67, wherein the patient received systemic chemotherapy.

73. The method of any of claim 9-12, 20-22, 31-34, 42-45, 54-57, or 65-67, wherein the cancer therapy is radiation therapy.

74. The method of claim 73, wherein the radiation therapy is cranial radiation therapy.

75. The method of any of claim 9-12, 20-22, 31-34, 42-45, 54-57, or 65-67, wherein the cancer therapy is surgery.

76. The method of claim 75, wherein the surgery is cranial surgery.

77. The method of any of claim 9-12, 20-22, 31-34, 42-45, 54-57, or 65-67, wherein the cancer therapy is a combination of chemotherapy and radiation therapy.

78. The method of any of claim 9-12, 20-22, 31-34, 42-45, 54-57, or 65-67, wherein the cancer therapy is a combination of at least two of the following: chemotherapy, radiation therapy, or surgery.

79. The method of any of claim 6, 17-19, 21, 22, 28, 39-41, 43-45, 51, 62-64, 66 or 67, wherein the patient is elderly.

80. The method of any of claim 6, 17-19, 21, 22, 28, 39-41, 43-45, 51, 62-64, 66, 67, or 79 wherein the patient is receiving concurrent treatment with a selective serotonin reuptake inhibitor, a serotonin norepinephrine reuptake inhibitor, a norepinephrine dopamine reuptake inhibitor, an atypical antidepressant, a tricyclic antidepressant, and/or a monoamine oxidase inhibitor.

81. The method of any of claim 6, 17-19, 21, 22, 28, 39-41, 43-45, 51, 62-64, 66, 67, 79, or 80 wherein the patient's inattention was inadequately treated with a selective serotonin reuptake inhibitor, a serotonin norepinephrine reuptake inhibitor, a norepinephrine dopamine reuptake inhibitor, an atypical antidepressant, a tricyclic antidepressant, and/or a monoamine oxidase inhibitor.

82. A method of treating fatigue, cognitive dysfunction, and/or inattention in a patient suffering from traumatic brain injury which comprises administering a therapeutically effective amount of an amphetamine-homoarginine conjugate or a salt of the amphetamine-homoarginine conjugate to a patient in need thereof.

83. A method of treating fatigue, cognitive dysfunction, and/or inattention in a menopausal patient, which comprises administering a therapeutically effective amount of an amphetamine-homoarginine conjugate or a salt of the amphetamine-homoarginine conjugate to a patient in need thereof.

84. A method of treating fatigue, cognitive dysfunction, and/or inattention in a patient suffering from the negative symptoms of schizophrenia, which comprises administering a therapeutically effective amount of an amphetamine-homoarginine conjugate or a salt of the amphetamine-homoarginine conjugate to a patient in need thereof.

85. A method of treating fatigue, cognitive dysfunction, and/or inattention in a patient suffering from diabetes mellitus type I or type II, which comprises administering a therapeutically effective amount of an amphetamine-homoarginine conjugate or a salt of the amphetamine-homoarginine conjugate to a patient in need thereof.

86. A method of treating cognitive dysfunction in a patient suffering from a cancer comprising administering a therapeutically effective amount of an amphetamine-homoarginine conjugate or a salt of the amphetamine-homoarginine conjugate to a patient in need thereof,
wherein the cognitive dysfunction is selected from the group consisting of: a short-term memory problem, an attention problem, a concentration problem, a planning problem, and a combination thereof; and
wherein the cognitive dysfunction appears before the patient receives chemotherapy.

87. A method of treating a cognitive dysfunction in a patient suffering from a cancer comprising administering a therapeutically effective amount of an amphetamine-homoarginine conjugate or a salt of the amphetamine-homoarginine conjugate to a patient in need thereof,
wherein the cognitive dysfunction is selected from the group consisting of: a short-term memory problem, an attention problem, a concentration problem, a planning problem, and a combination thereof; and
wherein the cognitive dysfunction arises during chemotherapy or after the patient receives chemotherapy.

88. A method of treating a wakefulness disorder which comprises administering a therapeutically effective amount of an amphetamine-homoarginine conjugate or a salt of the amphetamine-homoarginine conjugate to a patient in need thereof.

89. A method of treating narcolepsy in a patient which comprises administering a therapeutically effective amount of an amphetamine-homoarginine conjugate or a salt of the amphetamine-homoarginine conjugate to a patient in need thereof.

90. The method of claim 89, wherein the patient is a child patient or an adult patient.

91. A method of treating a wakefulness disorder caused by chemotherapy or radiation therapy which comprises administering a therapeutically effective amount of an amphetamine-homoarginine conjugate or a salt of the amphetamine-homoarginine conjugate to a patient in need thereof.

92. A method of treating a behavior caused by hyperactivity and impulsivity in a patient which comprises administering a therapeutically effective amount of an amphetamine-homoarginine conjugate or a salt of the amphetamine-homoarginine conjugate to a patient in need thereof.

93. A method of treating alcohol addiction in a patient which comprises administering a therapeutically effective amount of an amphetamine-homoarginine conjugate or a salt of the amphetamine-homoarginine conjugate to a patient in need thereof.

94. A method of treating smoking addiction in a patient which comprises administering a therapeutically effective amount of an amphetamine-homoarginine conjugate or a salt of the amphetamine-homoarginine conjugate to a patient in need thereof.

95. A method of treating symptoms of Fragile X syndrome in a patient which comprises administering a therapeutically effective amount of an amphetamine-homoarginine conjugate or a salt of the amphetamine-homoarginine conjugate to a patient in need thereof.

96. A method of treating a metabolic disorder in a patient which comprises administering a therapeutically effective amount of an amphetamine-homoarginine conjugate or a salt of the amphetamine-homoarginine conjugate to a patient in need thereof.

97. A method of treating obesity in a patient which comprises administering a therapeutically effective amount of an amphetamine-homoarginine conjugate or a salt of the amphetamine-homoarginine conjugate to a patient in need thereof.

98. A method of treating an appetite related symptom of the Prader Willi Syndrome in a patient which comprises administering a therapeutically effective amount of an amphetamine-homoarginine conjugate or a salt of the amphetamine-homoarginine conjugate to a patient in need thereof.

99. A method of treating Type 1 diabetes mellitus in a patient which comprises administering a therapeutically effective amount of an amphetamine-homoarginine conjugate or a salt of the amphetamine-homoarginine conjugate to a patient in need thereof.

100. A method of treating Type 2 diabetes mellitus in a patient which comprises administering a therapeutically effective amount of an amphetamine-homoarginine conjugate or a salt of the amphetamine-homoarginine conjugate to a patient in need thereof.

101. The method according to any one of claims 1-100 wherein the salt of the conjugate is mesylate, a hydrochloride, sulfate, oxalate, triflate, citrate, malate, tartrate, phosphate, nitrate, benzoate, or a mixture thereof.

102. The method according to any one of claims 1-101, wherein the amphetamine-homoarginine conjugate is 1-homoarginine-d-amphetamine dihydrochloride.

103. The method according to any one of claims 1-102, wherein the conjugate or the salt of the conjugate is in an amount of from about 1 mg to about 500 mg.

104. The method according to any one of claims 1-103, wherein the conjugate or the salt of the conjugate is in an amount of from about 5 mg to about 250 mg.

105. The method according to any one of claims 1-104, wherein the conjugate or the salt of the conjugate is in an amount of from about 10 mg to about 100 mg.
